(19) United States
(12) Patent Application Publication
(10) Pub. No.: US 2024/0357931 A1
(43) Pub. Date: Oct. 24, 2024

(54) ORGANIC ELECTRONIC DEVICES

(71) Applicant: Zhejiang Brilliant Optoelectronic Technology Co.,Ltd., Taizhou (CN)

(72) Inventors: Jiahui TAN, Taizhou (CN); Dizheng LIU, Taizhou (CN); Wenhan WU, Taizhou (CN); Junyou PAN, Taizhou (CN)

(73) Assignee: Zhejiang Brilliant Optoelectronic Technology Co.,Ltd., Taizhou (CN)

(21) Appl. No.: 18/756,263

(22) Filed: Jun. 27, 2024

Related U.S. Application Data
(63) Continuation of application No. PCT/CN2022/142368, filed on Dec. 27, 2022.

(30) Foreign Application Priority Data
Dec. 27, 2021 (CN) 202111613511.7

(51) Int. Cl. H10K 85/60; C07D 471/04; C07D 519/00; C09K 11/06; H10K 50/17

(52) U.S. Cl. CPC H10K 85/6572; C07D 471/04; C07D 519/00; C09K 11/06; H10K 85/654; C09K 2211/1018; H10K 50/171

(57) ABSTRACT

Disclosed are organic electronic devices including a cathode, an anode, at least two light-emitting layers disposed between the cathode and the anode, and a charge generation layer (CGL) disposed between the adjacent two light-emitting layers, the CGL includes an organic compound of formula (I). Also provided are compounds containing a structure of formula (1). Further provided are organic electronic devices comprising at least one compound.

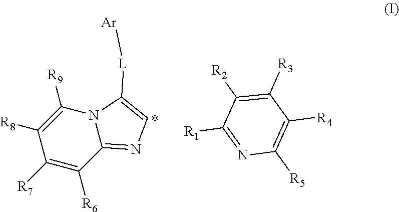

### CROSS-REFERENCE TO RELATED APPLICATIONS

The present application is a continuation of International Application No. PCT/CN2022/142368, filed on Dec. 27, 2022, which claims priority to Chinese Patent Application No. 202111613511.7, filed on Dec. 27, 2021. All of the aforementioned applications are incorporated herein by reference in their entireties.

### TECHNICAL FIELD

The present disclosure relates to the field of organic electronic device technology and material, and in particular to an organic light-emitting device with tandem structure.

### BACKGROUND

Due to the diversity of the synthesis, the low manufacturing cost, and the excellent optical and electrical properties, the organic light-emitting diodes (OLEDs) show great potential for optoelectronic device applications, such as flat-panel displays and lighting.

The organic electroluminescent phenomenon refers to a phenomenon of converting electrical energy to photonic energy with organic substances. An organic electroluminescent element utilizing the organic electroluminescent phenomenon usually has a structure comprising an anode, a cathode, and an organic layer therebetween. In order to improve the efficiency and lifetime of the organic electroluminescent element, the organic layer has a multi-layer structure, and each layer comprises different organic substances. Specifically, each layer can be a hole-injection layer, a hole-transport layer, a light-emitting layer, an electron-transport layer, an electron-injection layer, etc. When a voltage is applied between the two electrodes of such an organic electroluminescent element, holes are injected into the organic layer from the anode, and electrons are injected into the organic layer from the cathode; and an exciton is formed when an injected hole and an injected electron recombine in the emission layer. The exciton emits light when it transitions back to the ground state. The organic electroluminescent element has characteristics of self-emission, high luminance, high efficiency, low driving voltage, wide viewing angle, high contrast, high responsivity, etc. To fabricate OLED devices with lower driving voltage, higher luminous efficiency and longer device lifetime, the organic materials, the device architecture and the device fabrication processes need sustainable optimization.

Currently, tandem OLED devices, the OLED devices with muti-stacked light-emitting elements, are utilized to realize high luminance and fix the RGB color-mixing problems in large-size device. Generally, a charge generation layer (CGL) is disposed between the different light-emitting elements, to ensure that the charges can be effectively distributed to the sub-emitting elements, so that to improve the current efficiency of each sub-emitting element. This type of CGL is a PN junction, i.e., the N-type CGL and the P-type CGL are stacked in sequence. In such a CGL, due to the energy level difference between the N-type CGL and the P-type CGL, the charges are generated at the interfaces between the P-type CGL and the adjacent hole-injection/transport layer, resulting in the poor electron-injection efficiency of the N-type CGL. Meanwhile, the alkali or alkaline earth metals are doped in the N-type CGL, and the metals may diffuse into the the P-type CGL, thus leading to the shortened device lifetime. Furthermore, the thermal stability of the most common CGL materials also needs to be improved.

### SUMMARY

In one aspect, the present disclosure provides an organic electronic device comprising a cathode, an anode, at least two light-emitting layers disposed between the cathode and the anode, and a charge generation layer (CGL) disposed between the adjacent two light-emitting layers, where the CGL comprises an organic compound of formula (I):

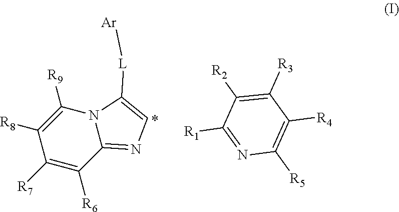

Where any one of R₁-R₅ is directly linked to * via a single bond; each of R₁-R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups can be fused to form a ring; L is a substituted/unsubstituted aromatic or heteroaromatic group containing 6 to 30 ring atoms; Ar is selected from a substituted/unsubstituted aromatic or heteroaromatic group containing 5 to 40 ring atoms, an aryloxy or heteroaryloxy group containing 5 to 40 ring atoms, or any combination thereof, where Ar may form a monocyclic or polycyclic aliphatic or aromatic ring systems with rings bonded thereto.

In another aspect, the present disclosure also provides a compound comprising a structure of formula (1):

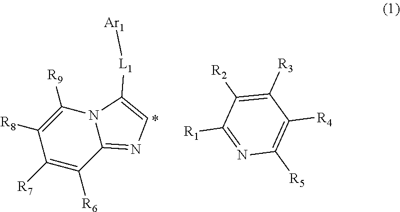

Where any one of R₁-R₅ is directly linked to * via a single bond; each of R₁ to R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups can be fused to form a ring; L₁ is a substituted/unsubstituted aromatic group containing 6 to 30 ring atoms; Ar₁ is selected from a substituted/unsubstituted aromatic or heteroaromatic group containing 5 to 40 ring atoms, an aryloxy or heteroaryloxy group containing 5 to 40 ring atoms, or any combination thereof, where Ar₁ may form a monocyclic or polycyclic aliphatic or aromatic ring systems with rings bonded thereto, and the ring atom of Ar₁ is directly linked to the ring atom of L₁ via a single bond.

In yet another aspect, the present disclosure further provides a polymer comprising at least one repeating unit, where the at least one repeating unit is selected from at least one structure of the compound as described herein.

In yet another aspect, the present disclosure further provides a formulation comprising an organic solvent, and at least one compound as described herein or at least one polymer as described herein.

In yet another aspect, the present disclosure further provides a mixture comprising a compound as described herein or a polymer as described herein, and at least one organic functional material, where the at least one organic functional material is selected from a hole-injection material, a hole-transport material, an electron-transport material, an electron-injection material, an electron-blocking material, a hole-blocking material, an emitting material (including a fluorescent emitting material, a phosphorescent emitting material, a thermally activated delayed fluorescence (TADF) material), or a host material.

In yet another aspect, the present disclosure further provides an organic electronic device comprising at least one compound as described herein or at least one polymer as described herein, where the organic electronic device is preferably selected from an organic light emitting diode, an organic photovoltaic cell, an organic light emitting electrochemical cell, an organic field effect transistor, an organic light emitting field effect transistor, an organic laser, an organic spintronic electronic device, an organic sensor, or an organic plasmon emitting diode.

Beneficial effect: the specific organic compounds of the present disclosure comprise nitrogen atoms with sp² hybrid orbitals and have relatively abundant electrons, thus the organic compounds have good electron-transport property and can be used as materials for electron-transport layers. Further, a gap state can be formed by bonding the nitrogen atoms of the organic compound to an alkali metal or alkaline earth metal compound, and the resulting reduced energy level difference between the N-type CGL and the P-type CGL facilitates the electron-injection into the N-type CGL, and realizes the electron transfer from the N-type CGL to the adjacent electron-transport layer. In addition, the N-containing organic compound can coordinate with the alkali metal or alkaline earth metal compound in the N-type CGL, preventing the alkali metal or alkaline earth metal compound from diffusing into the P-type CGL, thereby effectively prolonging the lifetime of the organic light-emitting diodes.

### DETAILED DESCRIPTION OF THE EMBODIMENTS

The present disclosure provides an organic electronic device comprising an organic compound, where the organic electronic device comprising the organic compound has low voltage and high device efficiency, resulting from the high electron injection capability, high electron mobility and high thermal stability of the organic compound. The present disclosure also provides a synthetic method of the organic compound, a structure of the organic electronic device comprising the organic compound, and a preparation method therefor. In order to make the objects, the technical solutions and the effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink”, and “ink” have the same meaning, and they are interchangeable with each other.

As used herein, the terms “aromatic group”, “aromatic”, and “aromatic ring system” have the same meaning, and they are interchangeable with each other.

As used herein, the terms “heteroaromatic group”, “heteroaromatic”, and “heteroaromatic ring system” have the same meaning, and they are interchangeable with each other.

As used herein, the term “substituted” means that a hydrogen atom of the compound is substituted.

As used herein, the term “alkali metal” refers to a Group I element of the periodic table. For example, an alkali metal may be lithium (Li), sodium (Na), potassium (K), rubidium (Rb), or cesium (Cs).

As used herein, the term “alkaline earth metal” refers to a Group II element of the periodic table. For example, an alkaline earth metal may be magnesium (Mg), calcium (Ca), strontium (Sr), or barium (Ba).

As used herein, “the number of ring atoms” means that the number of atoms constituting the ring itself of a structural compound (e. g., a monocyclic compound, a fused ring compound, a cross-linked compound, a carbocyclic compound, and a heterocyclic compound) by covalent bonding. When the ring is substituted with a substituent, the atoms contained in the substituent are not included in the ring atoms. The above rule applies for all cases without further specfic description. For example, the number of ring atoms of a benzene ring is 6, the number of ring atoms of a naphthalene ring is 10, and the number of ring atoms of a thienyl group is 5.

As used herein, the term “aromatic ring system” or “aromatic group” refers to a hydrocarbon group containing an aromatic ring, including monocyclic groups and polycyclic systems. The term “heteroaromatic ring system” or “heteroaromatic group” refers to a hydrocarbon group (containing a heteroatom) consisting of at least one heteroaromatic ring, including monocyclic groups and polycyclic systems. The heteroatoms are preferably selected from Si, N, P, O, S and/or Ge, particularly preferably selected from Si, N, P, O and/or S. The polycyclic systems contain two or more rings, in which two carbon atoms are shared by adjacent two rings, i. e., fused rings. Specifically, at least one of the rings in the polycyclic rings are aromatic or heteroaromatic. For the purposes of the present disclosure, the aromatic or heteroaromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic groups for the purposes of this invention.

Specifically, examples of aromatic groups include, but not limited to benzene, naphthalene, anthracene, phenanthrene, perylene, tetracene, pyrene, benzopyrene, triphenylene, acenaphthylene, fluorene, and derivatives thereof.

Specifically, examples of heteroaromatic groups include, but not limited to furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, pyrimidine, quinazoline, quinazolinone, and derivatives thereof.

As used herein, the term “alkyl group” may refer to a linear, branched and/or cyclic alkyl group. The carbon number of the alkyl group may be 1 to 50, 1 to 30, 1 to 20, 1 to 10, or 1 to 6. Non-limiting examples of alkyl groups include, but not limited to a methyl group, an ethyl group, a n-propyl group, an iso-propyl, a n-butyl group, a sec-butyl group, a tert-butyl group, an iso-butyl group, a 2-ethylbutyl group, a 3,3-dimethylbutyl group, a n-pentyl group, an iso-pentyl group, a neo-pentyl group, a tert-pentyl group, a cyclopentyl group, a 1-methylpentyl group, a 3-methylpentyl group, a 2-ethylpentyl group, a 4-methyl-2-pentyl group, a n-hexyl group, a 1-methylhexyl group, a 2-ethylhexyl group, a 2-butylhexyl group, a cyclohexyl group, a 4-methylcyclohexyl group, a 4-tert-butylcyclohexyl group, a n-heptyl group, a 1-methylheptyl group, a 2,2-dimethylheptyl group, a 2-ethylheptyl group, a 2-butylheptyl group, a n-octyl group, a tert-octyl group, a 2-ethyloctyl group, a 2-butyloctyl group, a 2-hexyloctyl group, a 3,7-dimethyloctyl group, a cyclooctyl group, a n-nonyl group, a n-decyl group, an adamantyl group, a 2-ethyldecyl group, a 2-butyldecyl group, a 2-hexyldecyl group, a 2-octyldecyl group, a n-undecyl group, a n-dodecyl group, a 2-ethyldodecyl group, a 2-butyldodecyl group, a 2-hexyldodecyl group, a 2-octyldodecyl group, a n-tridecyl group, a n-tetradecyl group, a n-pentadecyl group, a n-hexadecyl group, a 2-ethylhexadecyl group, a 2-butylhexadecyl group, a 2-hexylhexadecyl group, a 2-octylhexadecyl group, a n-heptadecyl group, a n-octadecyl group, a n-nonadecyl group, a n-eicosyl group, a 2-ethyleicosyl group, a 2-butyleicosyl group, a 2-hexyleicosyl group, a 2-octyleicosyl group, a n-heneicosyl group, a n-docosyl group, a n-tricosyl group, a n-tetracosyl group, a n-pentacosyl group, a n-hexacosyl group, a n-heptacosyl group, a n-octacosyl group, a n-nonacosyl group, a n-triacontyl, etc.

In one aspect, the present disclosure provides an organic electronic device comprising a cathode, an anode, at least two light-emitting layers disposed between the cathode and the anode, and a CGL disposed between the adjacent two light-emitting layers, where the CGL comprises an organic compound of formula (I):

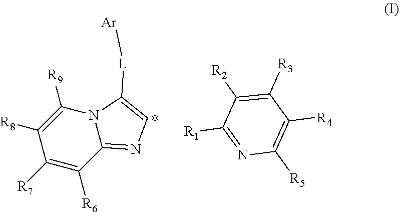

Where any one of R₁-R₅ is directly linked to * via a single bond; each of R₁ to R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups can be fused to form a ring; L is a substituted/unsubstituted aromatic or heteroaromatic group containing 6 to 30 ring atoms; Ar is selected from a substituted/unsubstituted aromatic or heteroaromatic group containing 5 to 40 ring atoms, an aryloxy or heteroaryloxy group containing 5 to 40 ring atoms, or any combination thereof, where Ar may form a monocyclic or polycyclic aliphatic or aromatic ring systems with rings bonded thereto.

In some embodiments, R₆-R₇ and R₈-R₉ may not both form a fused ring structure.

In some embodiments, each of R₁ to R₉ at each occurrence is independently selected from -D, a cyano group, a C₁-C₁₈ linear alkyl group, a C₃-C₁₈ branched/cyclic alkyl or alkoxy or thioalkoxy or silyl group, or a substituted/unsubstituted aromatic or heteroaromatic or aryloxy or heteroaryloxy group containing 5 to 30 ring atoms. In some embodiments, each of R₁ to R₉ at each occurrence is independently selected from -D, a C₁-C₁₂ linear alkyl group, or a substituted/unsubstituted aromatic or heteroaromatic or aryloxy or heteroaryloxy group containing 5 to 20 ring atoms. In some embodiments, each of R₁ to R₉ at each occurrence is independently selected from -D, a C₁-C₆ linear alkyl group, or a substituted/unsubstituted aromatic or heteroaromatic or aryloxy or heteroaryloxy group containing 5 to 15 ring atoms.

In some embodiments, R₁-R₉ of the organic compound at each occurrence are independently fully or partially deuterated.

In some embodiments, the organic compound comprises a structure of formula (II):

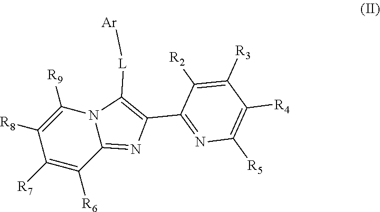

Where R₂-R₉, L, and Ar are identically defined as described herein.

In some embodiments, the organic compound comprises a structure of formula (II-a):

In some embodiments, the organic compound comprises a structure of CN formulas (III-1)-(III-6):

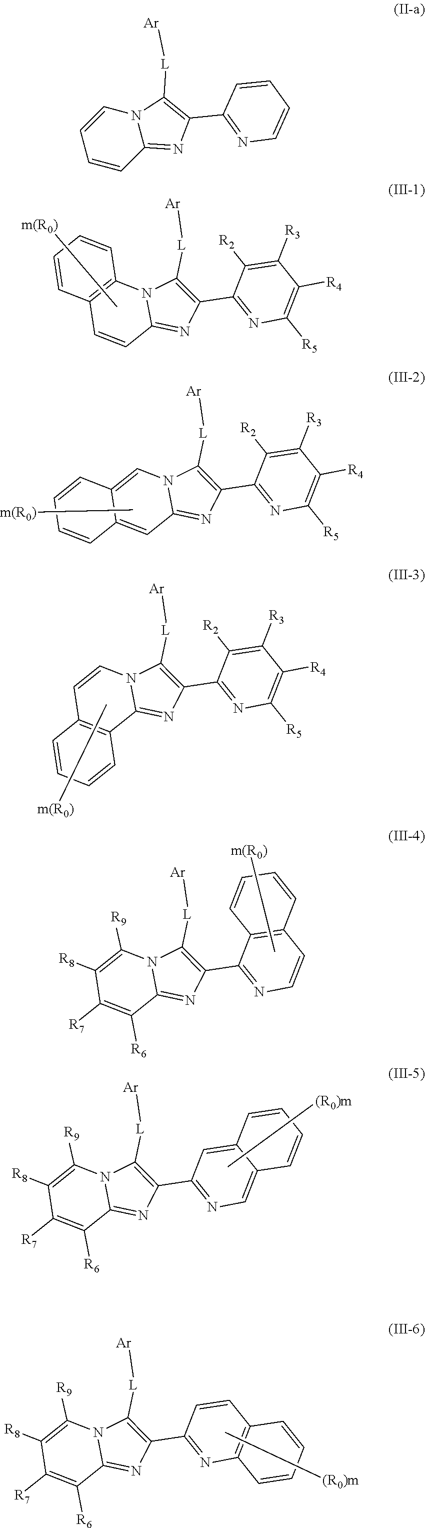

Where R₂-R₉, L, and Ar are identically defined as described herein; each R₀ is identically defined as R₁, m is an integer from 0 to 6.

In some embodiments, each L is independently selected from the following groups:

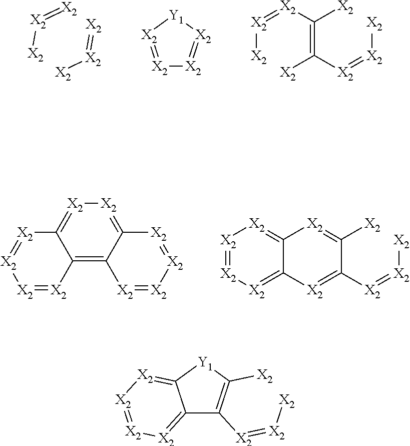

Where X₂ at each occurrence is CR₁₀ or N; Y₁ at each occurrence is independently selected from NR₁₁, CR₁₂R₁₃, O, S, SiR₁₄R₁₅, S═O, or SO₂; each of R₁₀ to R₁₅ at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, -Cl, -Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof.

In some embodiments, each L is independently selected from the following groups or any combination thereof:

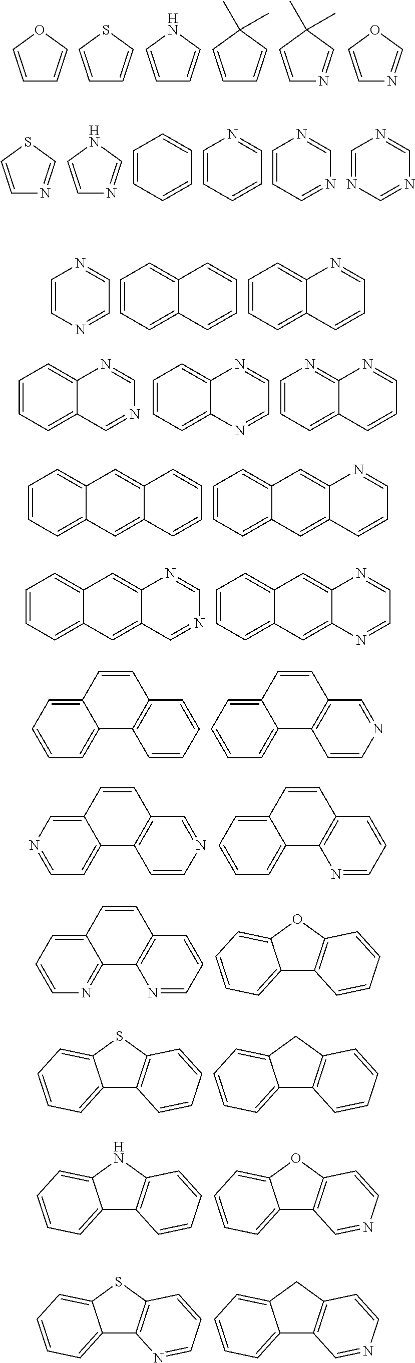

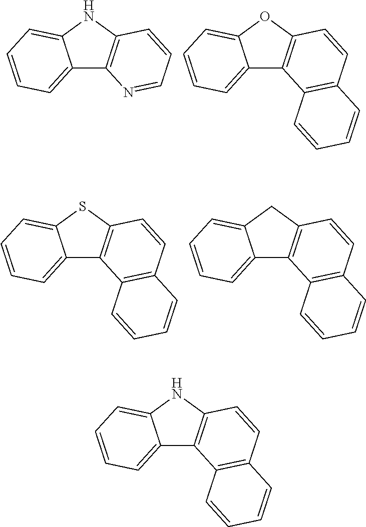

Where H atoms on the ring may be further substituted.

Further, each L is preferably selected from the following groups or any combination thereof:

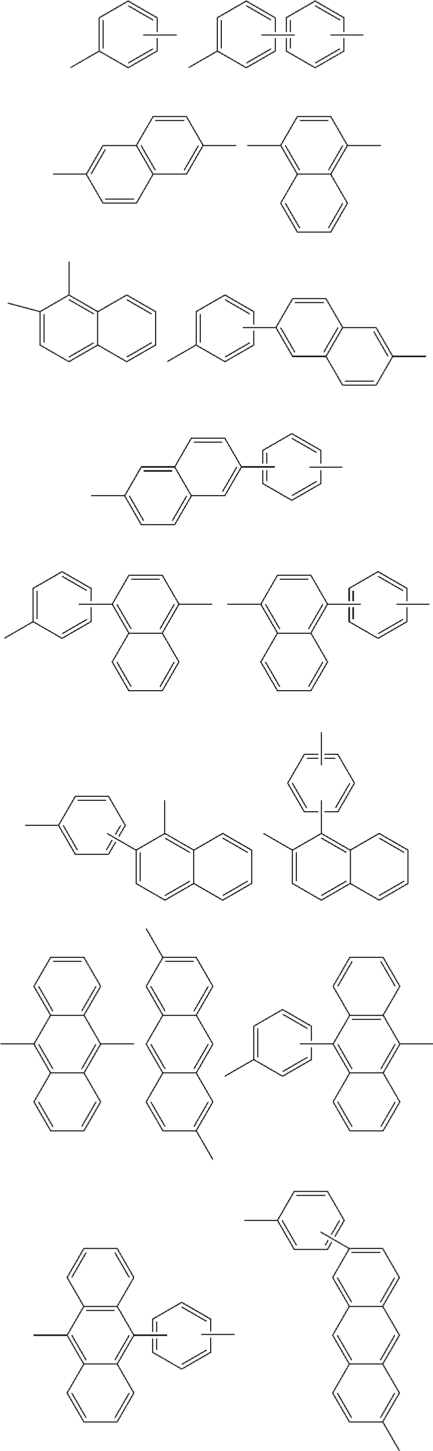

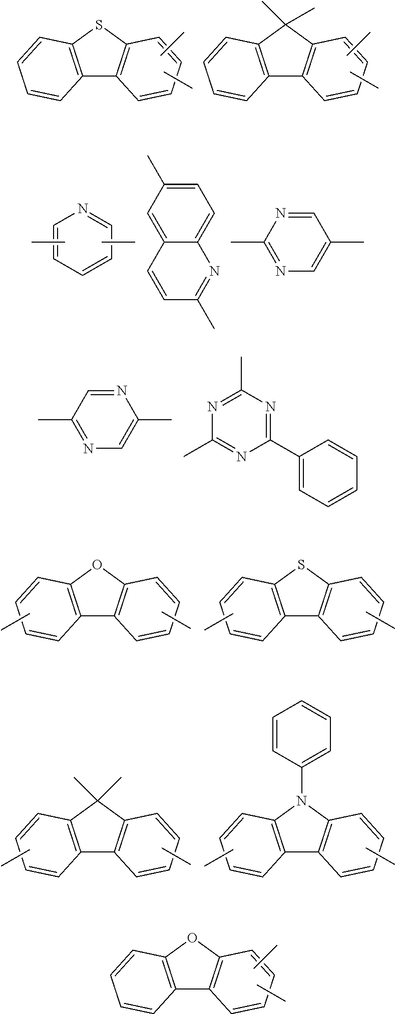

In some embodiments, Ar is independently selected from a substituted/unsubstituted aromatic or heteroaromatic or aryloxy or heteroaryloxy group containing 5 to 40 ring atoms, or any combination thereof, where one or more Ars may form a monocyclic or polycyclic aliphatic or aromatic ring system with each other and/or with the rings bonded thereto.

In some embodiments, Ar is independently selected from a substituted/unsubstituted aromatic or heteroaromatic group containing 5 to 20 ring atoms, a deuterated/undeuterated aryloxy or heteroaryloxy group containing 5 to 20 ring atoms, or any combination thereof, where one or more Ars may form a monocyclic or polycyclic aliphatic or aromatic ring system with each other and/or with the rings bonded thereto.

In some embodiments, Ar is independently selected from a substituted/unsubstituted aromatic or heteroaromatic group containing 5 to 15 ring atoms, a deuterated/undeuterated aryloxy or heteroaryloxy group containing 5 to 15 ring atoms, or any combination thereof, where one or more Ars may form a monocyclic or polycyclic aliphatic or aromatic ring system with each other and/or with the rings bonded thereto.

In some embodiments, Ar is selected from benzene, naphthalene, phenanthrene, triphenylene, biphenyl, terphenyl, or a structure in which one or more carbon atoms of these structures are substituted with N atoms. In this case, the metal coordination ability of these organic compounds is strong, and the N-type CGL of the tandem devices as described herein is more suitable for employing the above-mentioned organic compound.

As used herein, the term “aromatic group” refers to a hydrocarbon group consisting of an aromatic ring, including monocyclic groups and polycyclic systems. The term “heteroaromatic group” refers to a hydrocarbon group (containing a heteroatom) consisting of at least one heteroaromatic ring, including monocyclic groups and polycyclic systems. The polycyclic systems contain two or more rings, in which two carbon atoms are shared by two adjacent rings, i. e., fused rings. Specifically, at least one of the rings in the polycyclic rings are aromatic or heteroaromatic. For the purposes of the present disclosure, the aromatic or heteroaromatic groups contain not only aromatic or heteroaromatic systems, but also have a plurality of aromatic or heteroaromatic groups linked by short non-aromatic units (for example C, N, O, Si, S, P atoms). Therefore, a system such as 9,9′-spirobifluorene, 9,9-diarylfluorene, triarylamine, diaryl ether, and the like is also considered to be aromatic groups for the purposes of this invention.

Specifically, examples of aromatic groups include, but not limited to benzene, naphthalene, anthracene, phenanthrene, perylene, tetracene, pyrene, benzopyrene, triphenylene, acenaphthylene, fluorene, and derivatives thereof.

Specifically, examples of heteroaromatic groups include, but not limited to furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, pyrimidine, quinazoline, quinazolinone, and derivatives thereof.

In some embodiments, Ar comprises an electron-withdrawing group, which is more suitably employed in the electron-transport layer of the organic electronic device.

In some embodiments, in the organic compound of the organic electronic device as described herein, the electron-withdrawing group contained in Ar may be selected from F, a cyano group, one of the following groups, or any combination thereof:

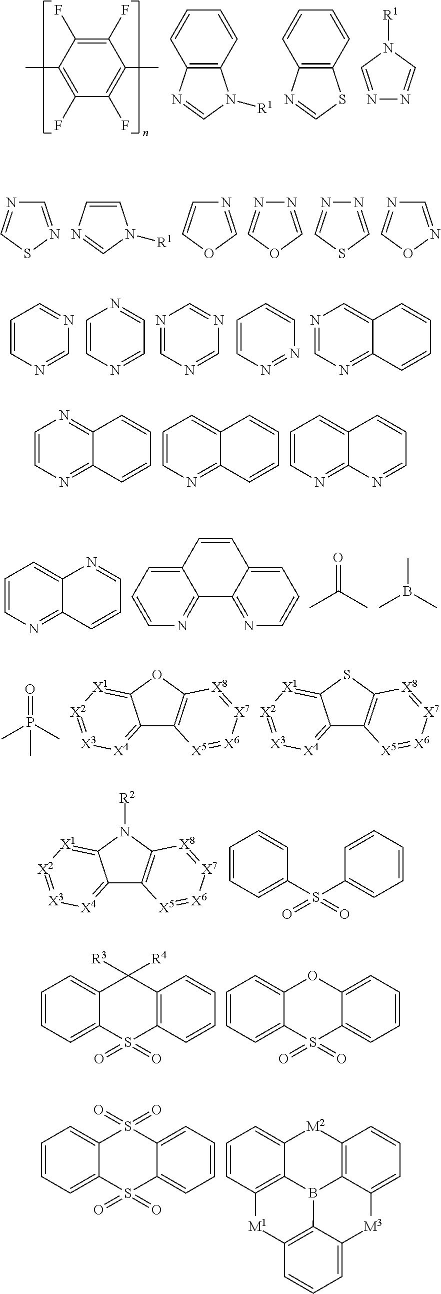

Where n is 1, or 2, or 3; each of X¹ to X⁸ is CR⁵ or N, and at least one of them is N; meanwhile any two adjacent positions can form a monocyclic or polycyclic aliphatic or aromatic ring system; M¹, M², M³ independently represent N(R⁶), C(R⁷)₂, Si(R⁸)₂, O, C═N(R⁹), C═C(R¹⁰)₂, P(R¹¹), P(═O)R¹², S, S═O, SO₂, or null; R¹-R¹² are identically defined as the above-mentioned R₁₀.

In some embodiments, in the organic compound of the organic electronic device as described herein, the electron-withdrawing group is selected from one of the following groups or any combination thereof:

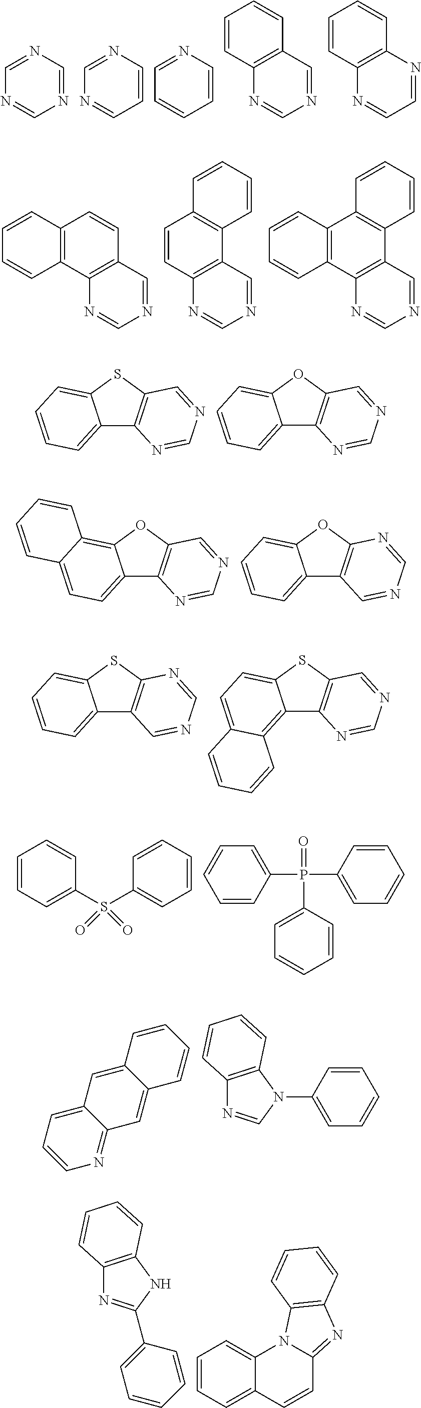

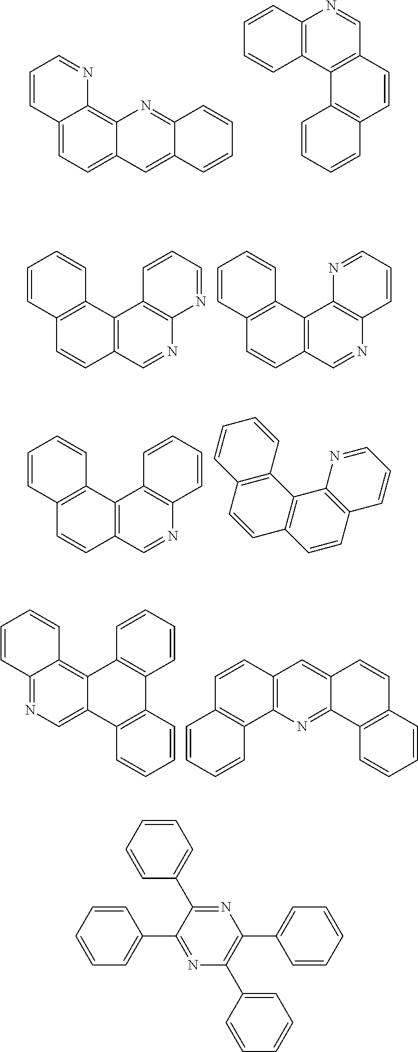

The above-mentioned groups may optionally be substituted with 0, or 1, or 2, or 3 substituents selected from D, F, Cl, Br, a cyano group, a C₁-C₄ alkyl group, a C₁-C₃ haloalkyl group, a phenyl group, a naphthyl group, a fluorenyl group, a spirofluorenyl group, or a C₃-C₁₀ cycloalkyl group.

In some embodiments, the organic compound of the organic electronic device is preferably selected from, but not limited to, the following structures:

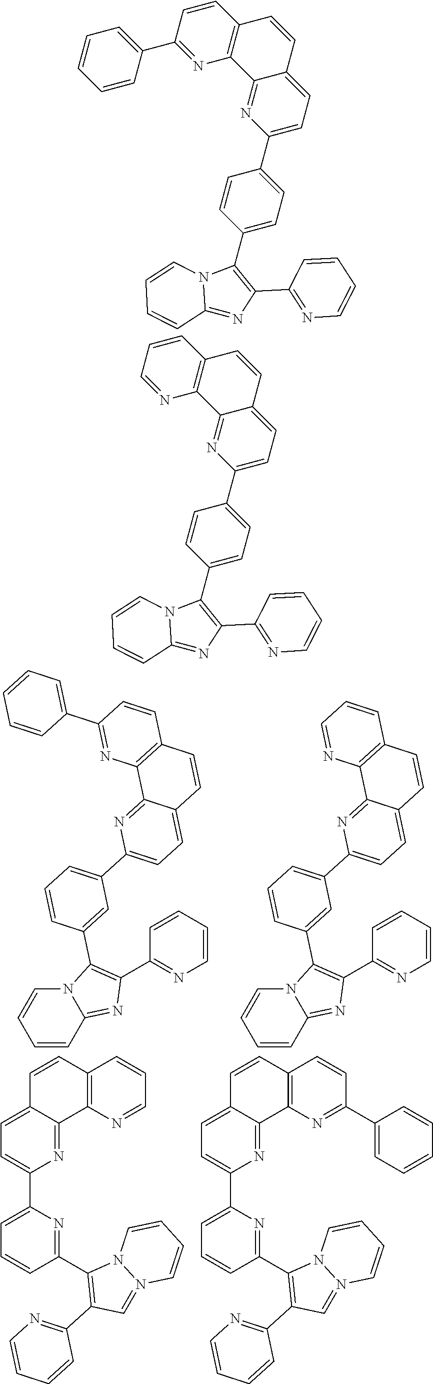

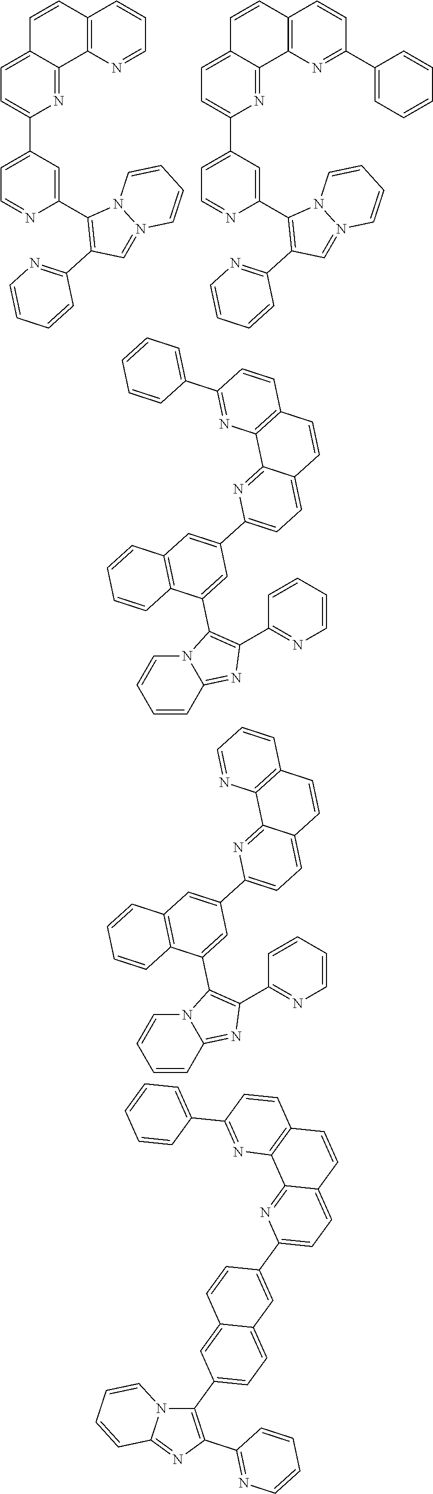

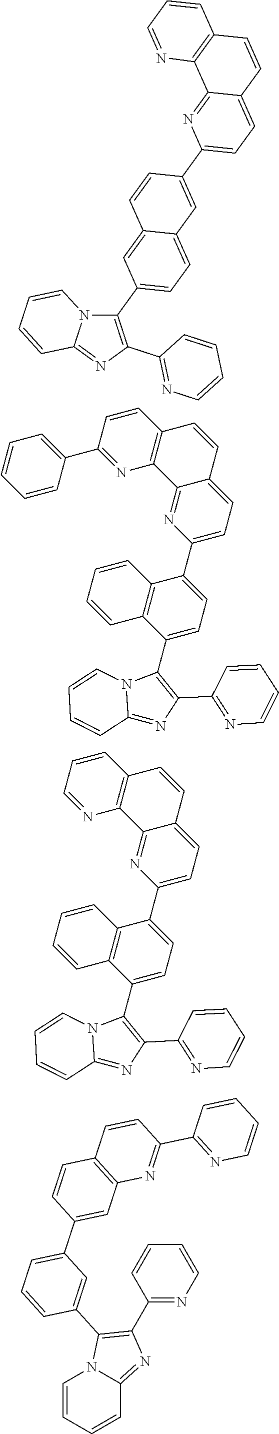

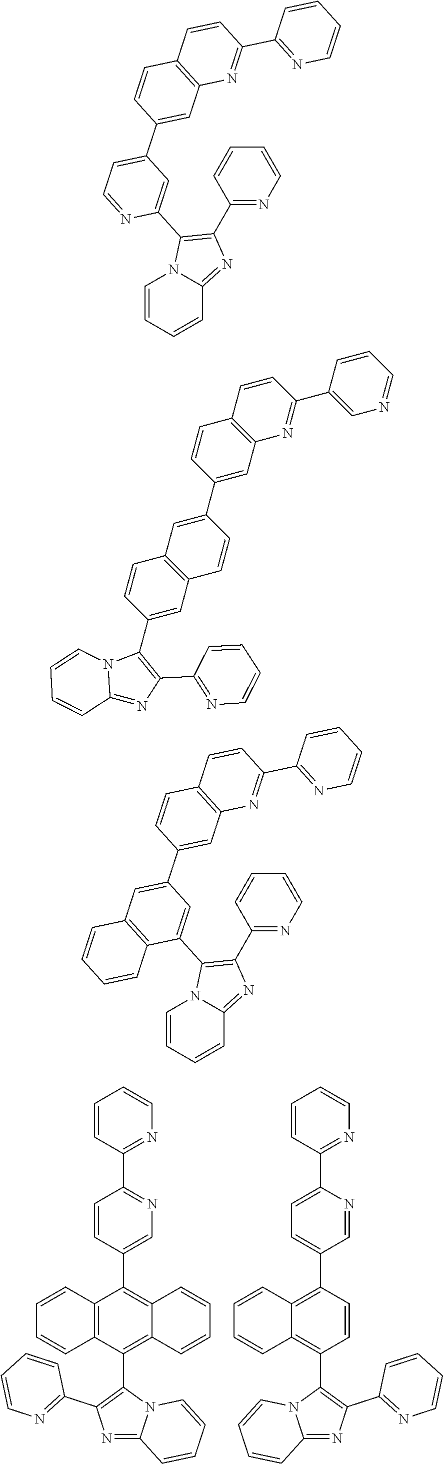

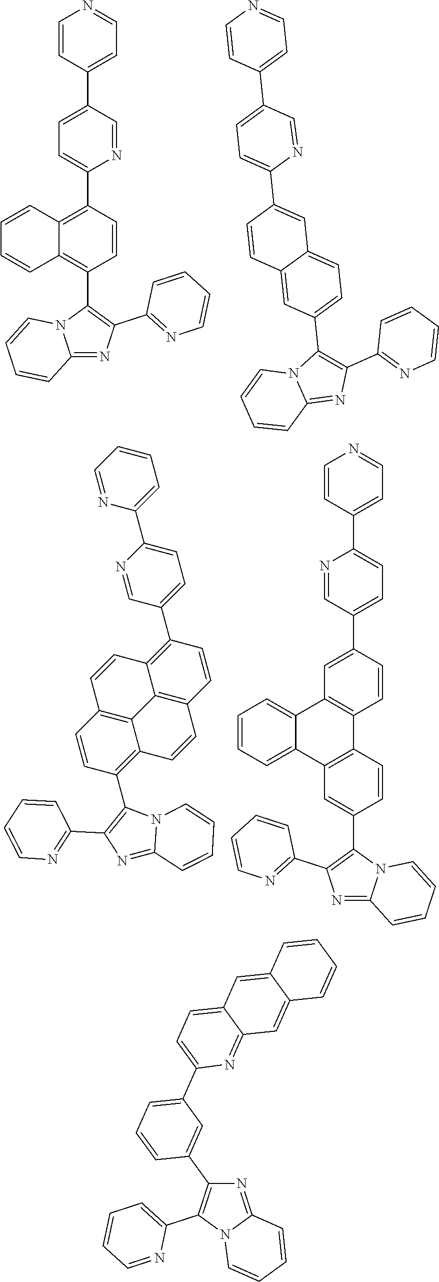

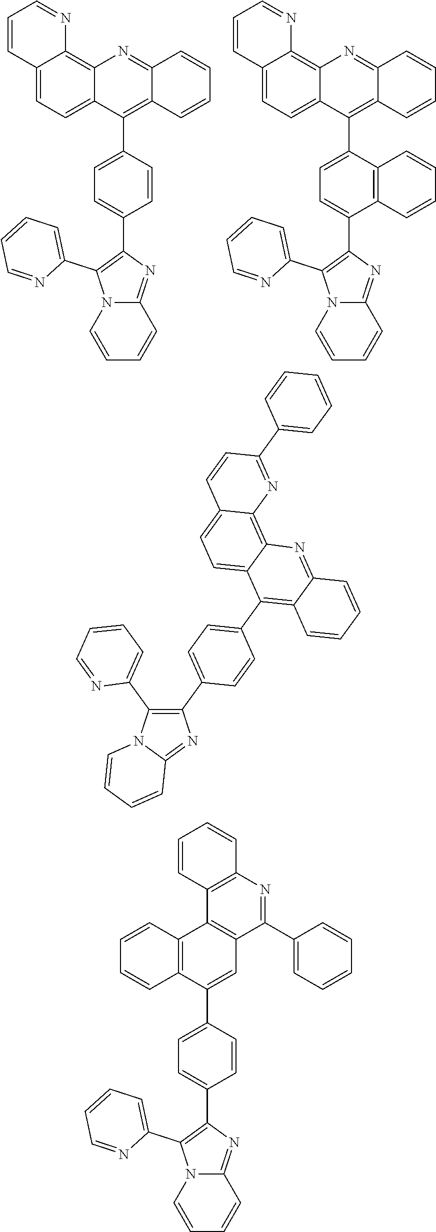

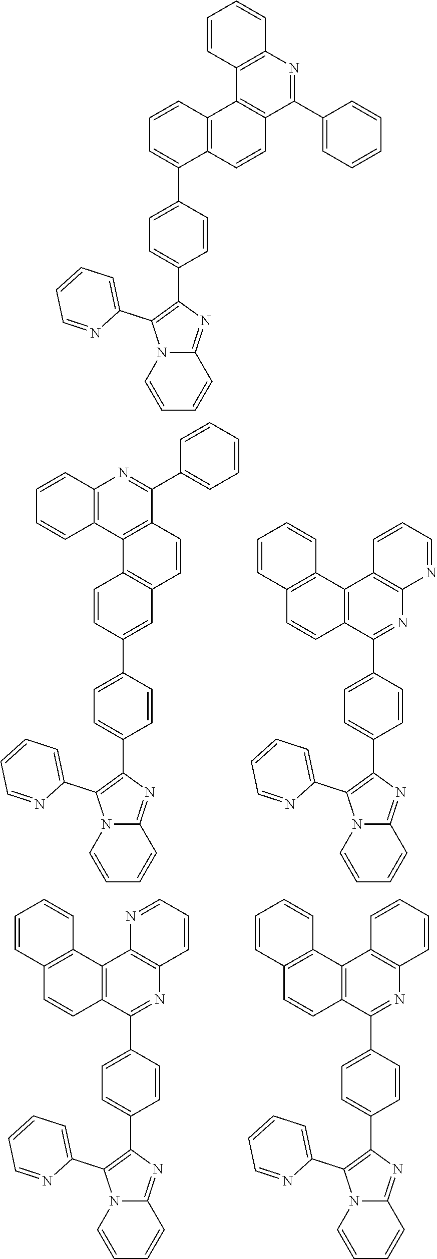

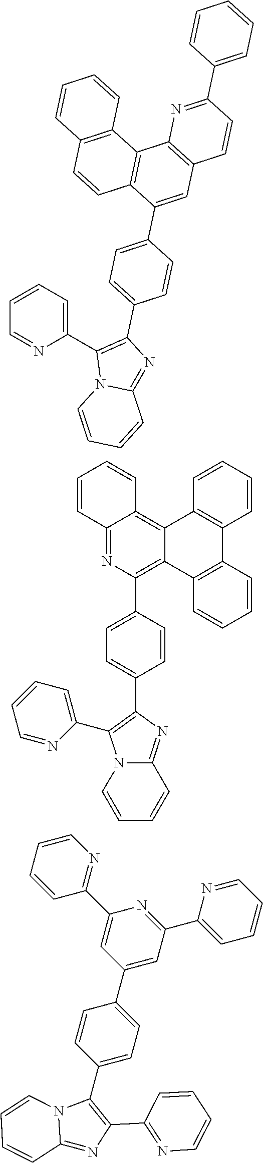

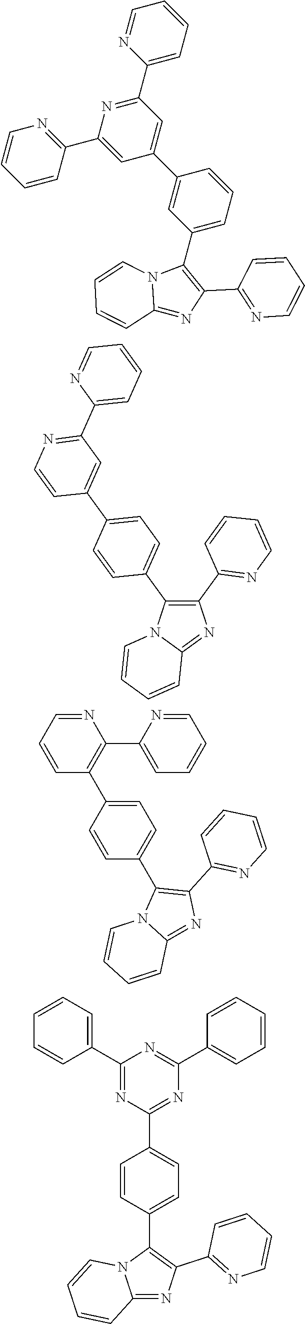

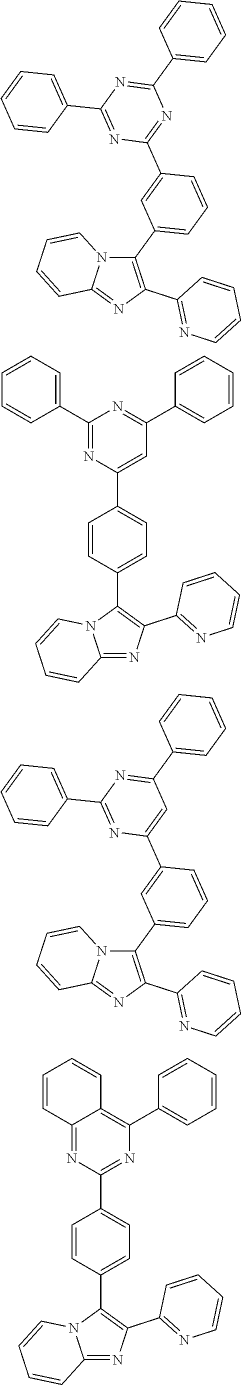

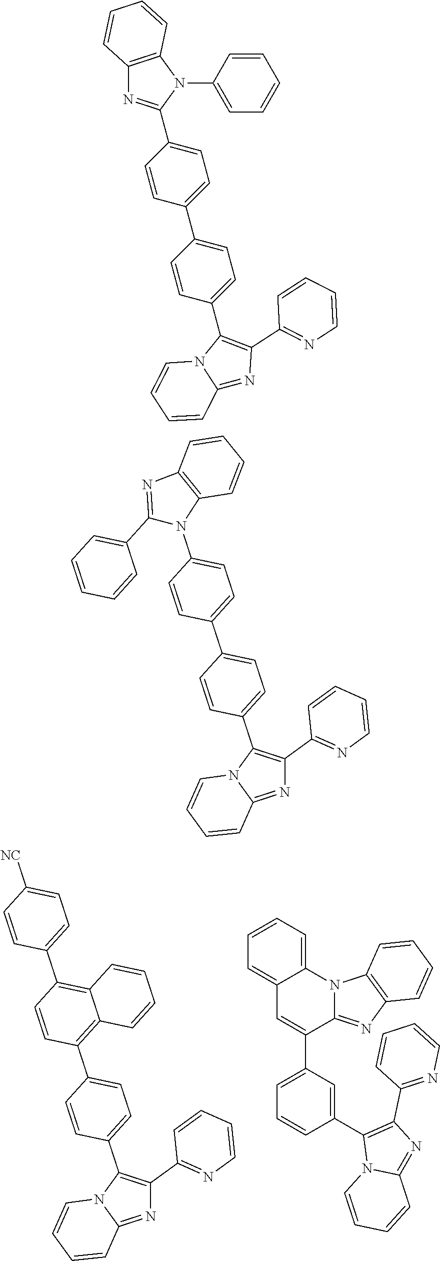

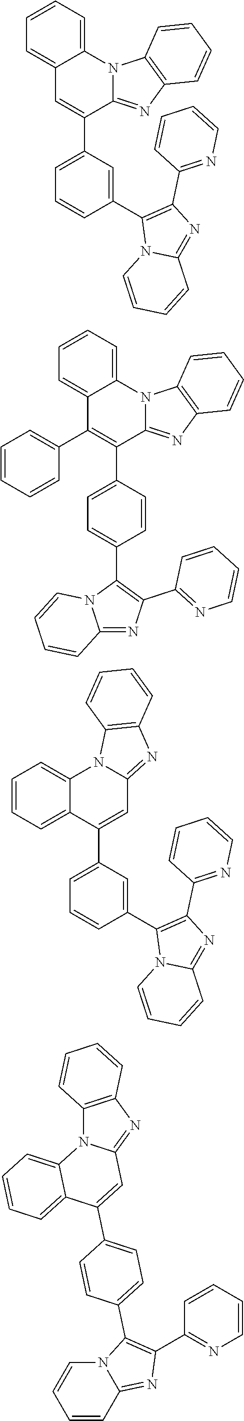

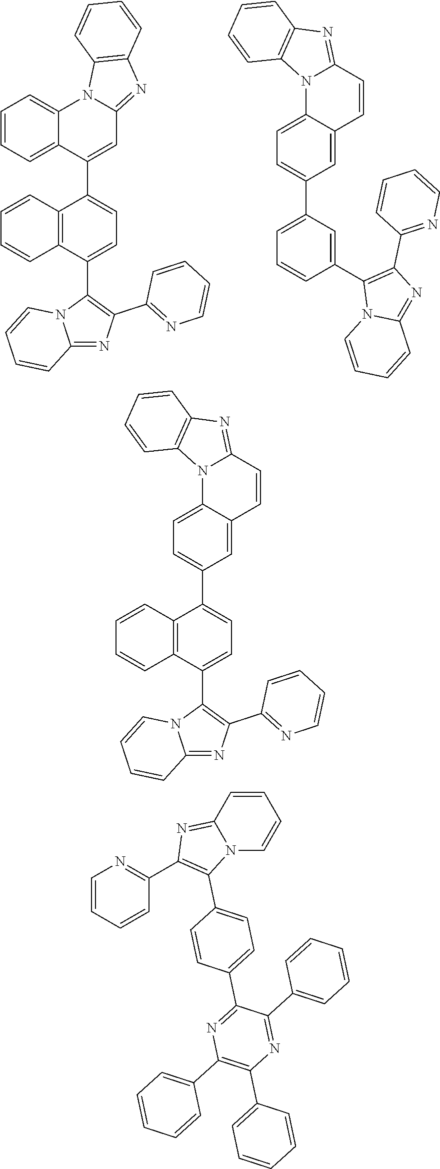

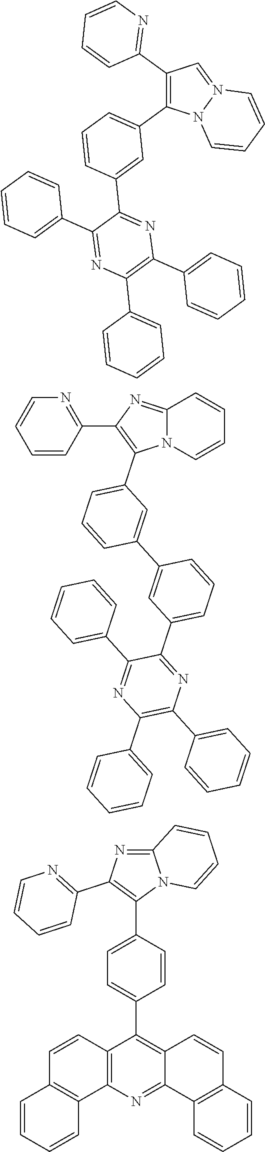

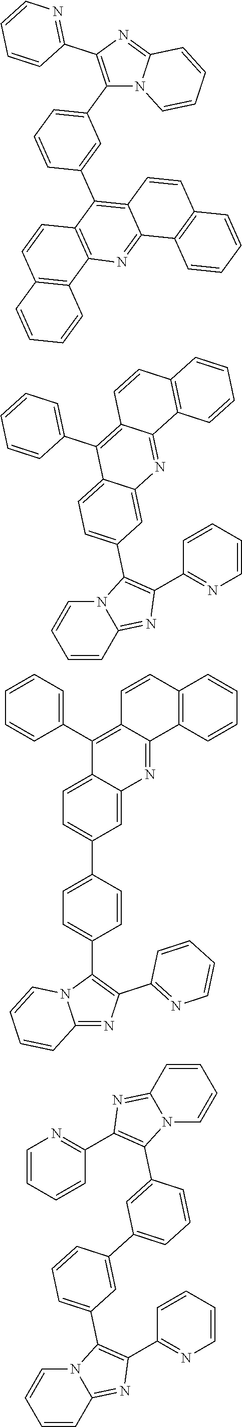

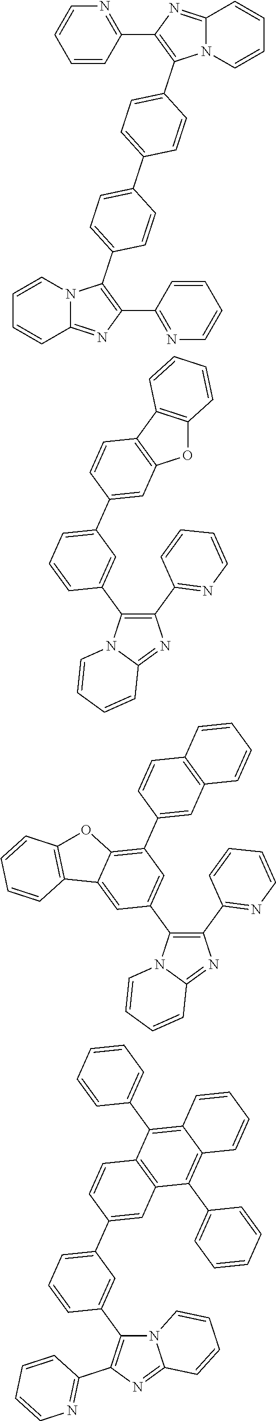

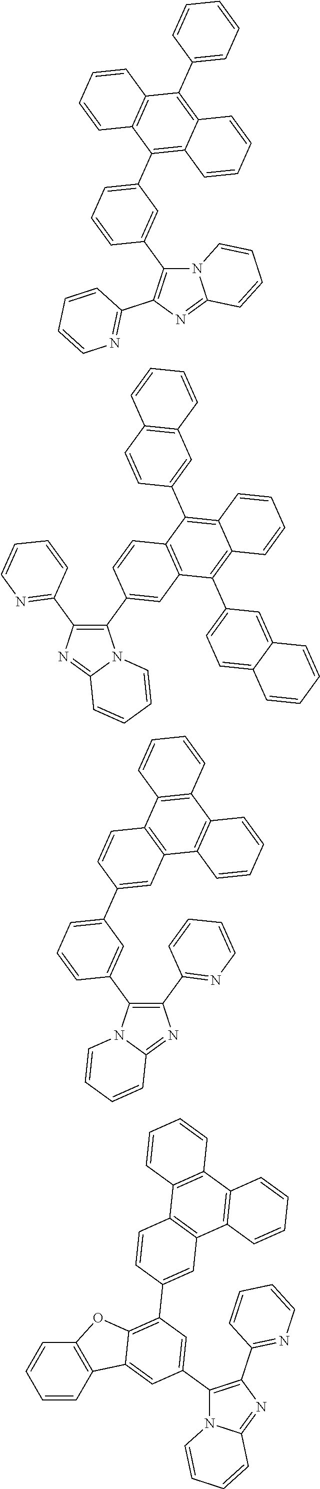

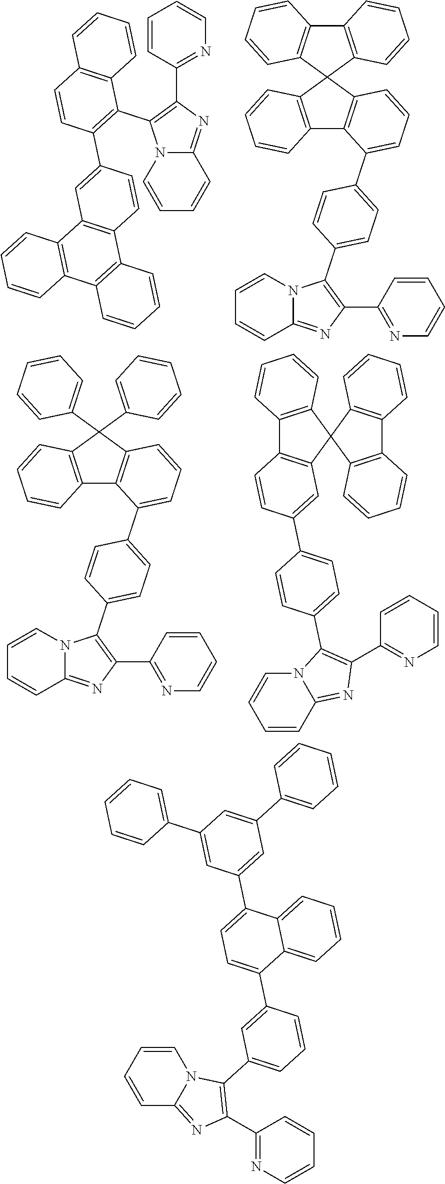

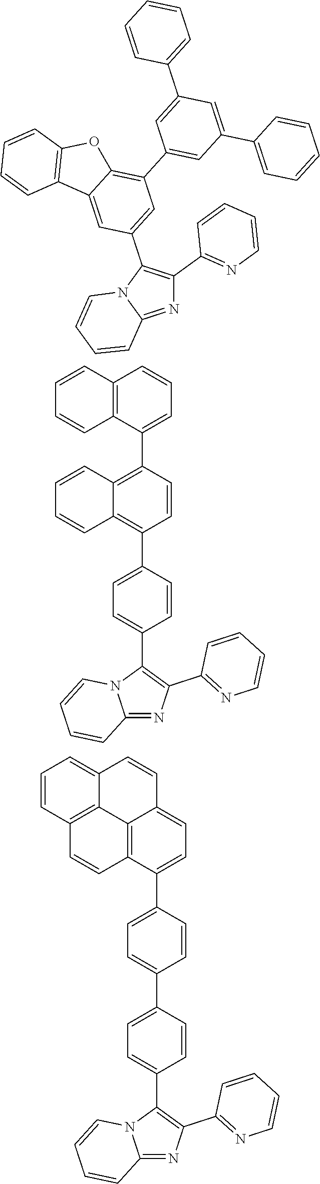

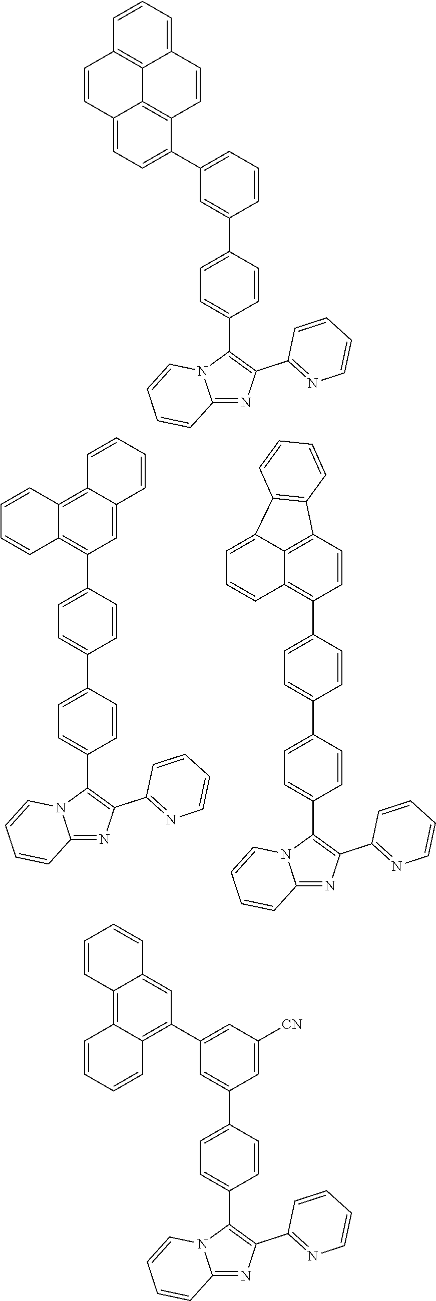

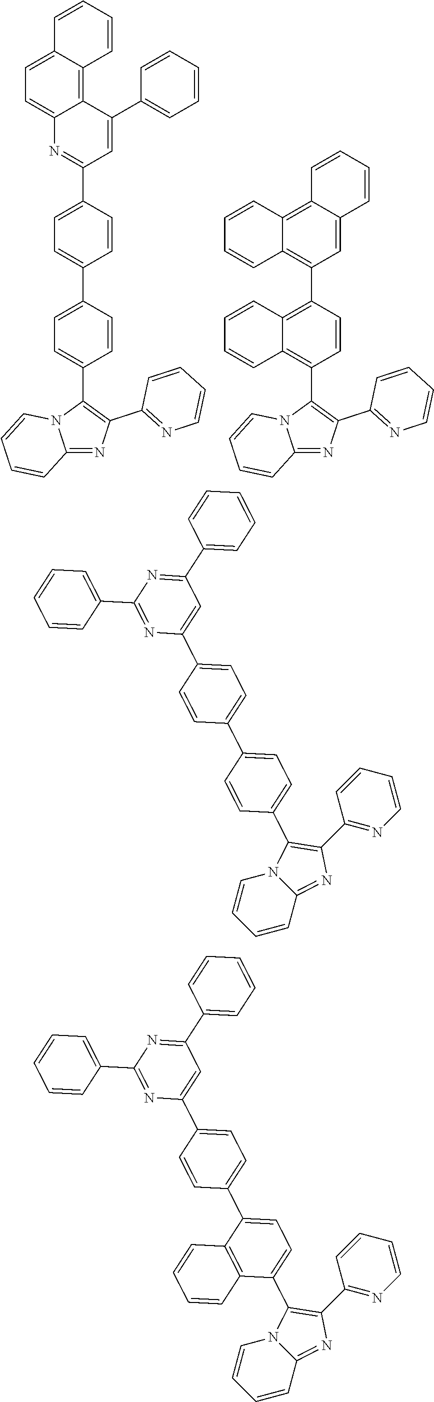

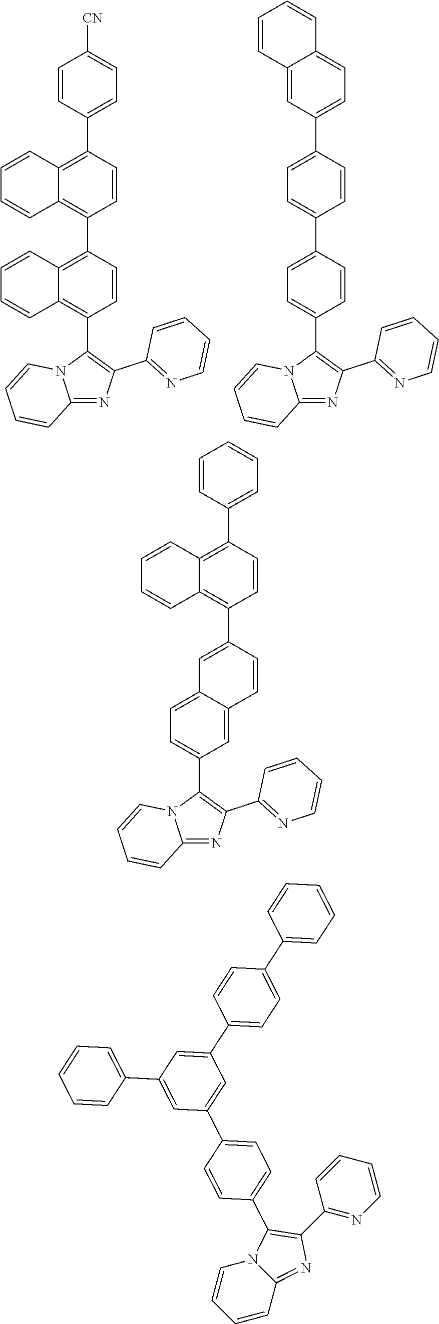

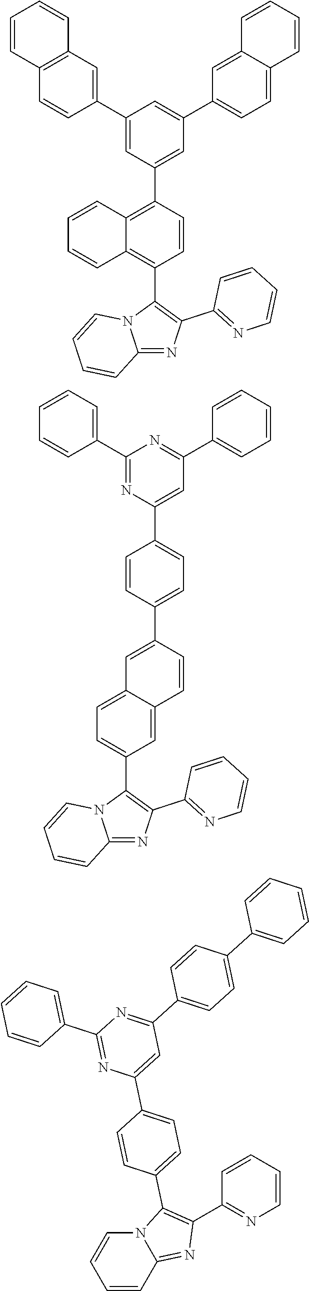

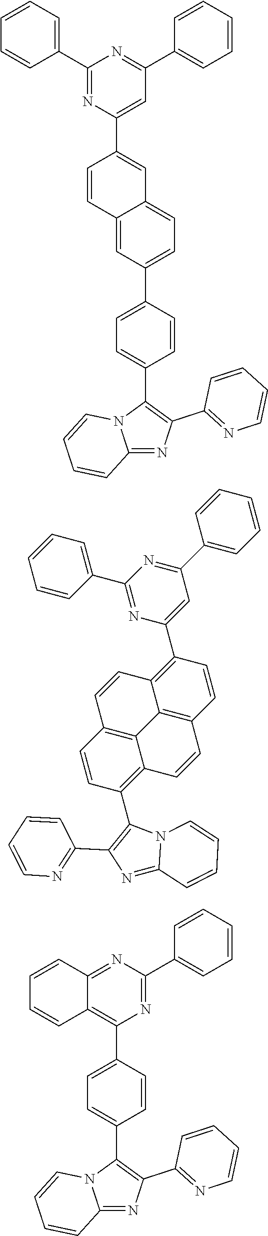

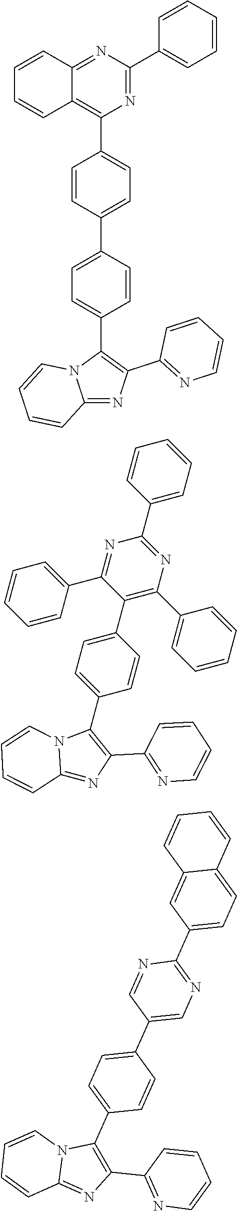

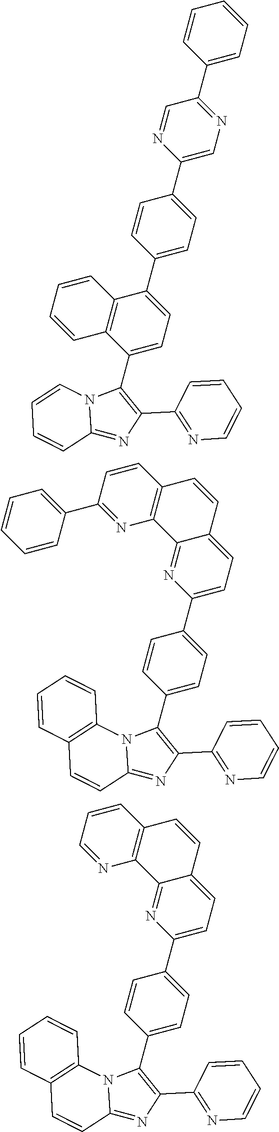

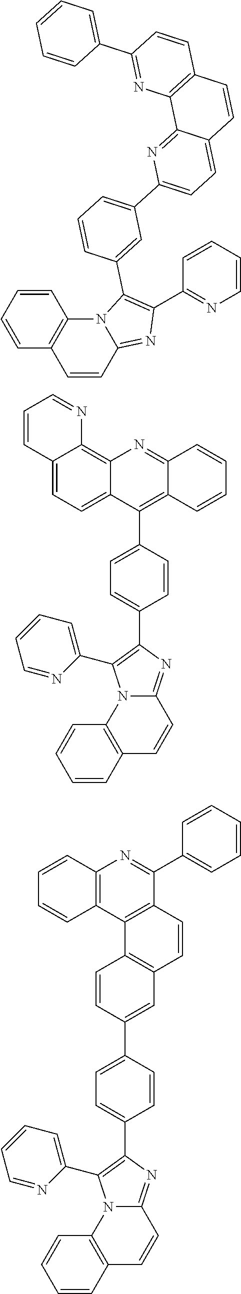

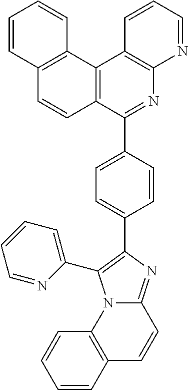 [SMILES: c1ccc(-c2c(-c3ccc(-c4nc5ncccc5c5c4ccc4ccccc45)cc3)nc3ccc4ccccc4n23)nc1]

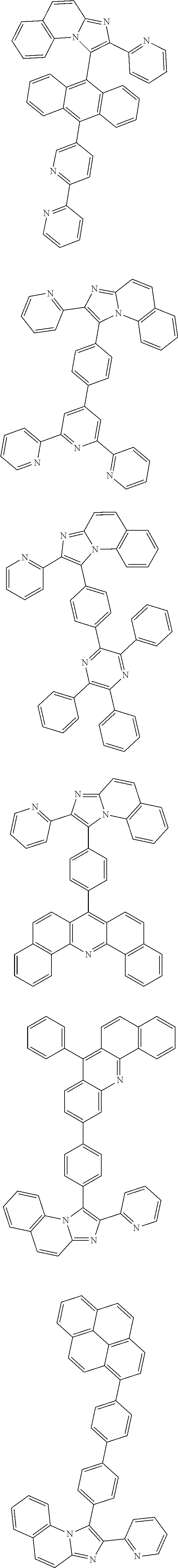

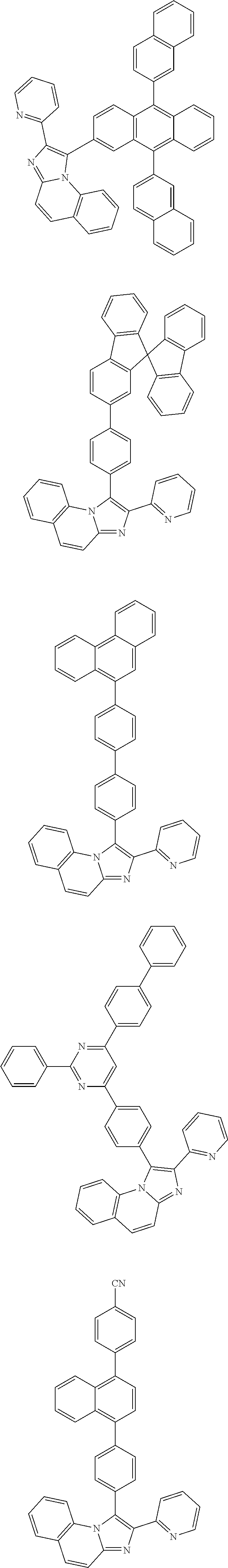 [SMILES: c1ccc(-c2nc3ccc4ccccc4n3c2-c2ccc3c(-c4ccc5ccccc5c4)c4ccccc4c(-c4ccc5ccccc5c4)c3c2)nc1 ; c1ccc(-c2nc3ccc4ccccc4n3c2-c2ccc(-c3ccc4c(c3)C3(c5ccccc5-c5ccccc53)c3ccccc3-4)cc2)nc1 ; c1ccc(-c2nc3ccc4ccccc4n3c2-c2ccc(-c3ccc(-c4cc5ccccc5c5ccccc45)cc3)cc2)nc1 ; c1ccc(-c2ccc(-c3cc(-c4ccc(-c5c(-c6ccccn6)nc6ccc7ccccc7n56)cc4)nc(-c4ccccc4)n3)cc2)cc1 ; N#Cc1ccc(-c2ccc(-c3ccc(-c4c(-c5ccccn5)nc5ccc6ccccc6n45)cc3)c3ccccc23)cc1]

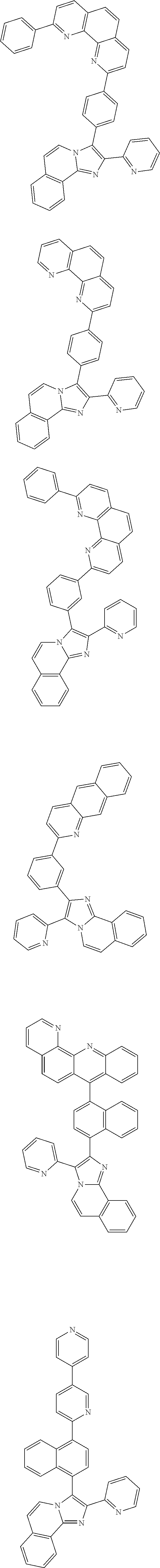

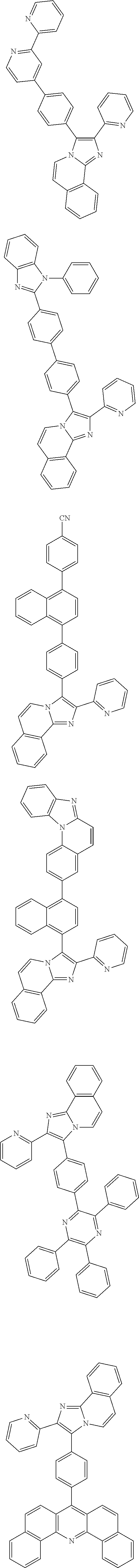

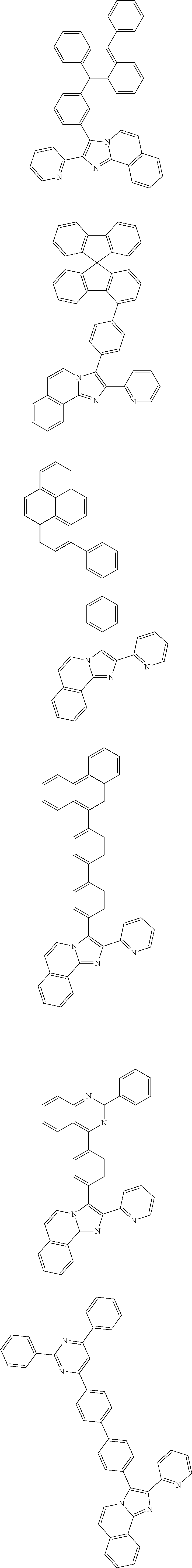 [SMILES: c1ccc(-c2c3ccccc3c(-c3cccc(-c4c(-c5ccccn5)nc5c6ccccc6ccn45)c3)c3ccccc23)cc1 ; c1ccc(-c2nc3c4ccccc4ccn3c2-c2ccc(-c3cccc4c3-c3ccccc3C43c4ccccc4-c4ccccc43)cc2)nc1 ; c1ccc(-c2nc3c4ccccc4ccn3c2-c2ccc(-c3cccc(-c4ccc5ccc6cccc7ccc4c5c67)c3)cc2)nc1 ; c1ccc(-c2nc3c4ccccc4ccn3c2-c2ccc(-c3ccc(-c4cc5ccccc5c5ccccc45)cc3)cc2)nc1 ; c1ccc(-c2nc(-c3ccc(-c4c(-c5ccccn5)nc5c6ccccc6ccn45)cc3)c3ccccc3n2)cc1 ; c1ccc(-c2cc(-c3ccc(-c4ccc(-c5c(-c6ccccn6)nc6c7ccccc7ccn56)cc4)cc3)nc(-c3ccccc3)n2)cc1]

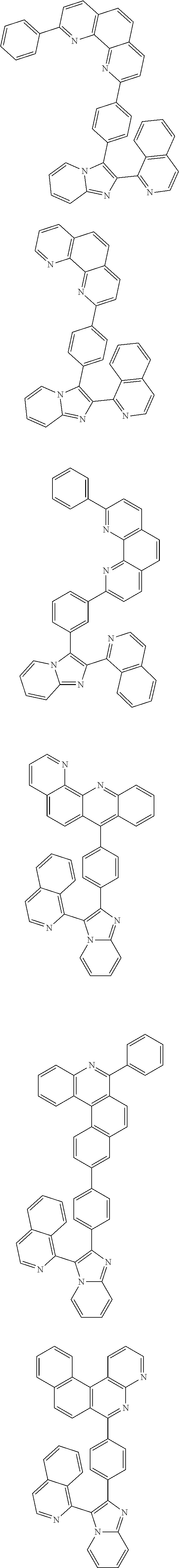

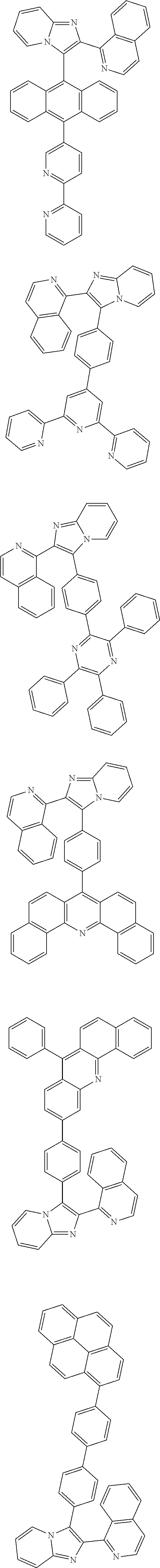

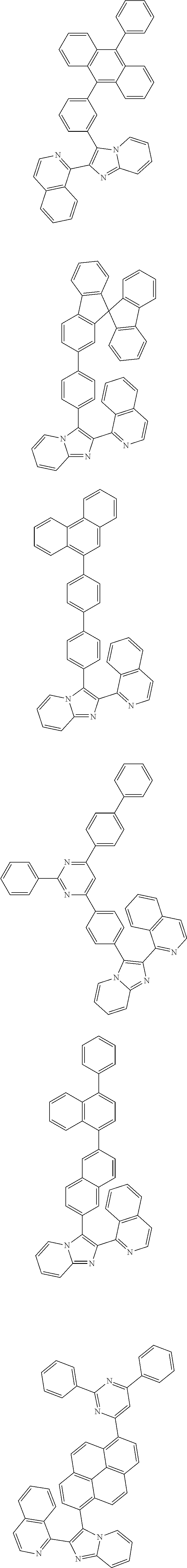

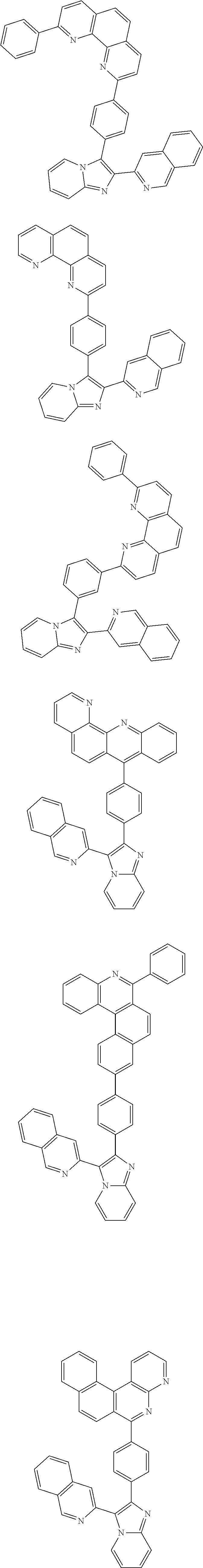

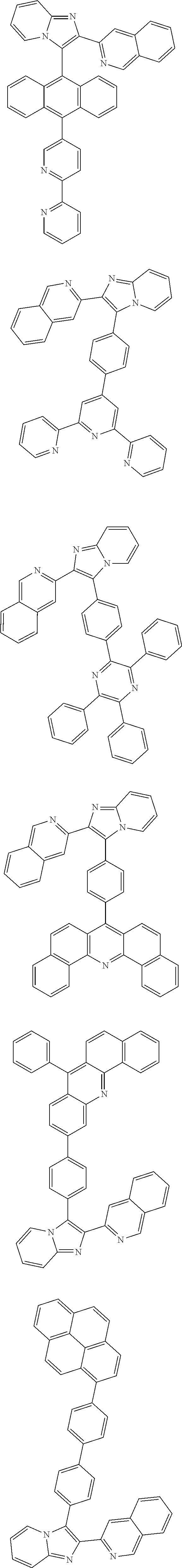

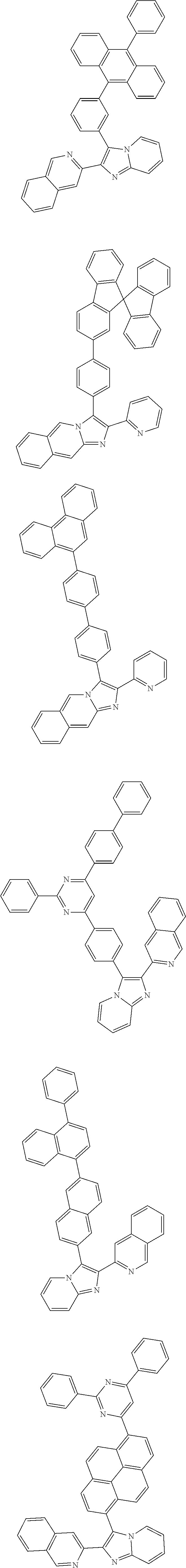

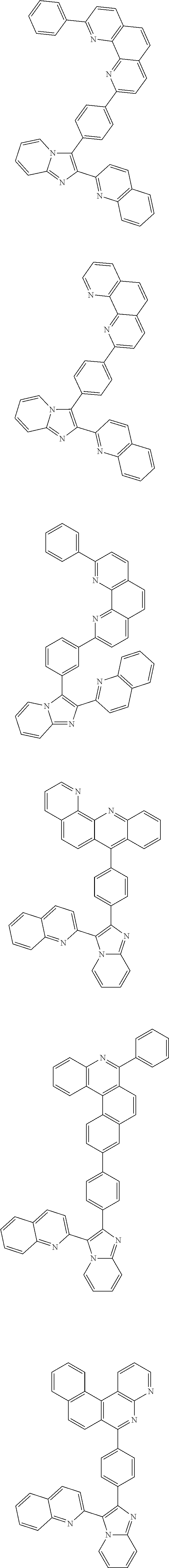 [SMILES: c1ccc(-c2ccc3ccc4ccc(-c5ccc(-c6c(-c7ccc8ccccc8n7)nc7ccccn67)cc5)nc4c3n2)cc1 ; c1ccc2nc(-c3nc4ccccn4c3-c3ccc(-c4ccc5ccc6cccnc6c5n4)cc3)ccc2c1 ; c1ccc(-c2ccc3ccc4ccc(-c5cccc(-c6c(-c7ccc8ccccc8n7)nc7ccccn67)c5)nc4c3n2)cc1 ; c1ccc2nc(-c3c(-c4ccc(-c5c6ccccc6nc6c5ccc5cccnc56)cc4)nc4ccccn34)ccc2c1 ; c1ccc(-c2nc3ccccc3c3c2ccc2cc(-c4ccc(-c5nc6ccccn6c5-c5ccc6ccccc6n5)cc4)ccc23)cc1 ; c1ccc2nc(-c3c(-c4ccc(-c5nc6ncccc6c6c5ccc5ccccc56)cc4)nc4ccccn34)ccc2c1]

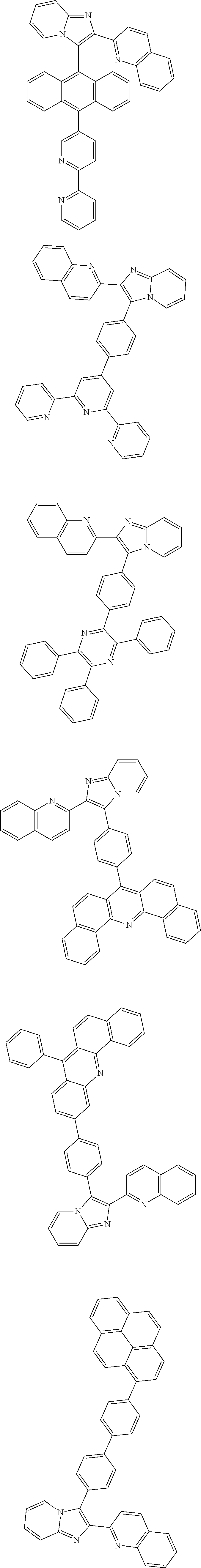

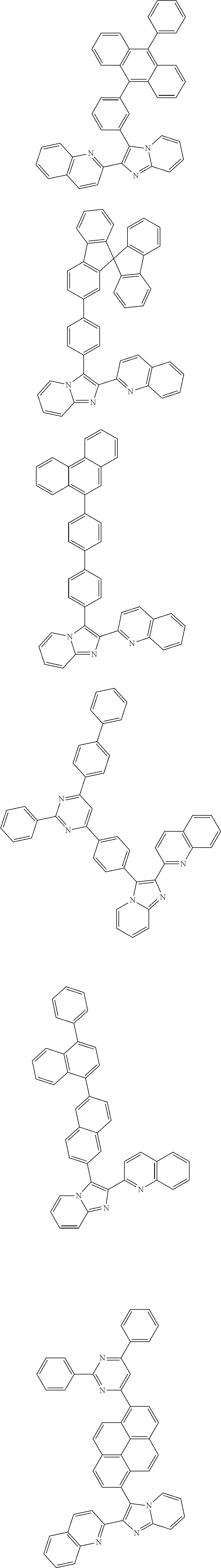

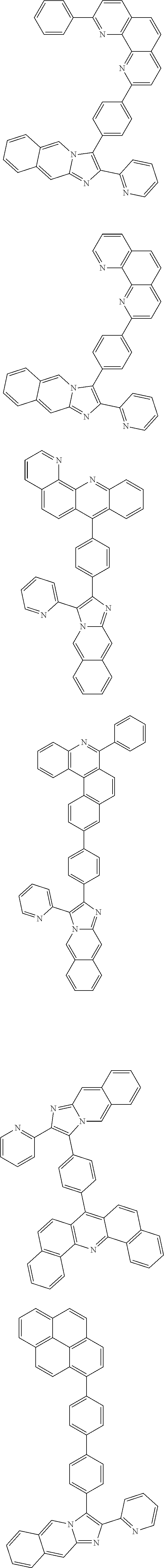

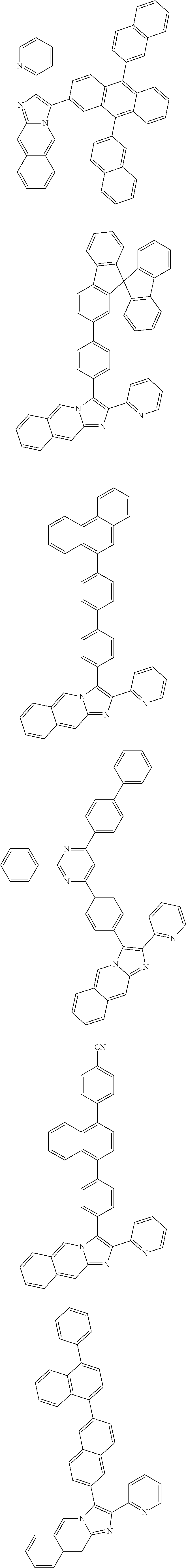

In addition, material applications can be diversified by introducing various substituents into the structure of formula (I) to synthesize compounds with unique properties of the introduced substituents, or by preparing them as monomer units to form polymers with fine control of the energy band gap, and by enhancing the properties at the interface between organic materials. By way of example, materials meeting the requirements for the organic material layer can be synthesized by introducing substituents (commonly used as hole-injection materials, hole-transport materials, emitting materials, electron-transport materials, and charge generation materials for organic light emitting equipment) into the core structure for use in the preparation of the corresponding functional layer of the organic electronic device.

The present disclosure also relates to a synthetic method of the organic compound with a structure of formula (I) using the reagents with active groups. These active reagents comprise at least one leaving group, such as a bromine, an iodine, a boronic acid, or a boronic ester. The appropriate C—C coupling reactions are familiar to the persons skilled in the art and are described in the literature, particularly appropriate and preferred coupling reactions are the SUZUKI, STILLE, and HECK coupling.

The CGL provides efficient carrier injection into the adjacent electroluminescent units, where metals, metal compounds, or other inorganic compounds are effective for carrier injection. However, these materials typically have low resistivity, leading to pixel crosstalk. In addition, the CGL should be as transparent as possible, so that the radiation from the electroluminescent units could be emitted out of the device. Therefore, organic materials are preferably utilized in the CGL. Generally, the CGL further includes n-type CGL and p-type CGL.

The n-type CGL also acts as the electron-transport layer and the electron-injection layer. The n-type CGL generally comprises an electron-transport material and a n-type dopant. In addition, the n-type dopant may be selected from an alkali metal, an alkaline-earth metal, an organic alkali metal complex, or an organic alkaline-earth metal complex. The metal doped in the n-type CGL may be further selected from a rare earth metal or a lanthanide metal. For example, a suitable metal may be preferably selected from any one of lithium (Li), sodium (Na), magnesium (Mg), calcium (Ca), cesium (Cs), or ytterbium (Yb).

In some embodiments, the amount of the metal doped in the n-type CGL is preferably from 1% to 10%, more preferably from 2% to 8%, of the total volume of the n-type CGL, in order to realize more efficient carrier (e.g., electrons or holes) transport to the adjacent stacks and prevent lateral leakage due to conductivity.

In some embodiments, the thickness of the n-type CGL is preferably set between 5 nm and 20 nm, more preferably between 10 nm and 15 nm. The thickness is also determined to prevent the lateral leakage of the n-type CGL and improve the carrier transport efficiency.

The alkali metals of the organic alkali metal complexes are elements that make up Group I of the periodic table, i.e., lithium, sodium, potassium, rubidium, cesium, where lithium is particularly preferred. Suitable organic alkali metal complexes include compounds described in U.S. Pat. No. 7,767,317B2, EP1941562B1, and EP1144543B1.

In some embodiments, the suitable organic alkali metal complexes comprise a structure of formula (A):

(M⁺)ₘ₁(Q)ₙ₁ (A)

Where M is an alkali metal, Q is an anionic organic ligand; m1 and n1 are integers and chosen independently for the electroneutrality of the above-mentioned complex.

The preferred anionic organic ligands (Q) should be monoanionic and contain at least one ionizable site consisting of oxygen, nitrogen, or carbon. In the case of enols or other oxygen-containing tautomers, lithium may be considered to be bonded to the oxygen atom as so drawn, and lithium may also be bonded elsewhere to form a chelate. In some embodiments, the ligand comprises at least one nitrogen atom that can form a coordinate bond or coordinate covalent bond with lithium. m1 and n1 are integers greater than 1.

In some embodiments, the suitable organic alkali metal complexes comprise a structure of formula (B):

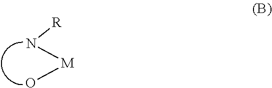

Where R at each occurrence is independently selected from the following groups: hydrogen, deuterium, halogen atoms (F, Cl, Br, I), cyano, alkyl, alkoxy, amino, alkenyl, alkynyl, aryl, heteroalkyl, aryl, or heteroaryl. The arc represents two, or three, or four atoms and the bonds, so as to form a five- or six- or seven-membered ring with the metal M when necessary, where the atoms may also be substituted with one or more R¹s, and M is an alkali metal selected from lithium, sodium, potassium, rubidium, or cesium.

The organic alkali metal complexes may be in the form of monomers, or aggregates as described herein, e.g., two alkali metal ions and two ligands, four alkali metal ions and four ligands, six alkali metal ions and six ligands, or other forms.

Preferred organic alkali metal complexes are compounds of the following formulas:

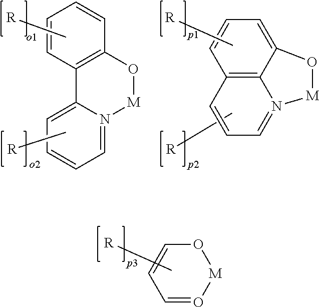

Where the symbols used are identically defined as described herein; additionally, R is identically defined as described herein; o1 and o2 are the same or different at each occurrence, and are integers from 0 to 4; p1, p2, and p3 are the same or different at each occurrence, and are integers from 0 to 3.

In some embodiments, the alkali metal (M) is selected from lithium, sodium, or potassium, more preferably is lithium or sodium, and most preferably is lithium.

In some embodiments, the suitable organic alkali metal complexes comprise a structure of formula (C):

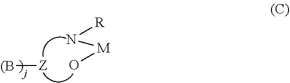

Where Z and the arc represent 2˜4 atoms and the bonds to form a five-, or six-, or seven-membered ring with the lithium cation; each B represents a hydrogen atom or an independent selected substituent of Z, preferably represents two or more substituents which can form a fused ring or a fused ring system; j is an integer from 0 to 3.

In some embodiments, the substituents R and B form an additional ring system. The additional ring system may further contain the additional heteroatoms forming the multidentate ligands by coordinating with lithium. The ideal heteroatoms are nitrogen or oxygen. Begley disclosed compounds of formula (C) in U.S. Pat. No. 2006086405, which is specially incorporated herein by reference. In formula (C), the oxygen atom is preferably part of a hydroxyl group, a carboxyl group, or a ketone group. Suitable examples of nitrogen-containing ligands include 8-hydroxyquinoline, 2-(hydroxymethyl)pyridine, piperidinecarboxylic acid, or 2-picolinic acid.

Suitable examples of organic alkali metal complexes those can be used as n-type dopants are listed below:

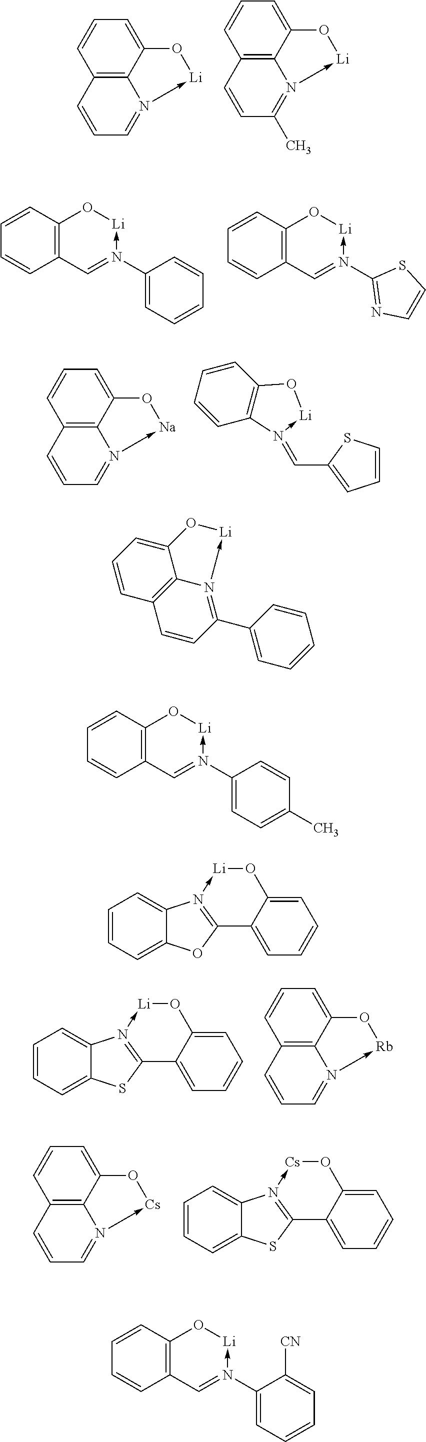

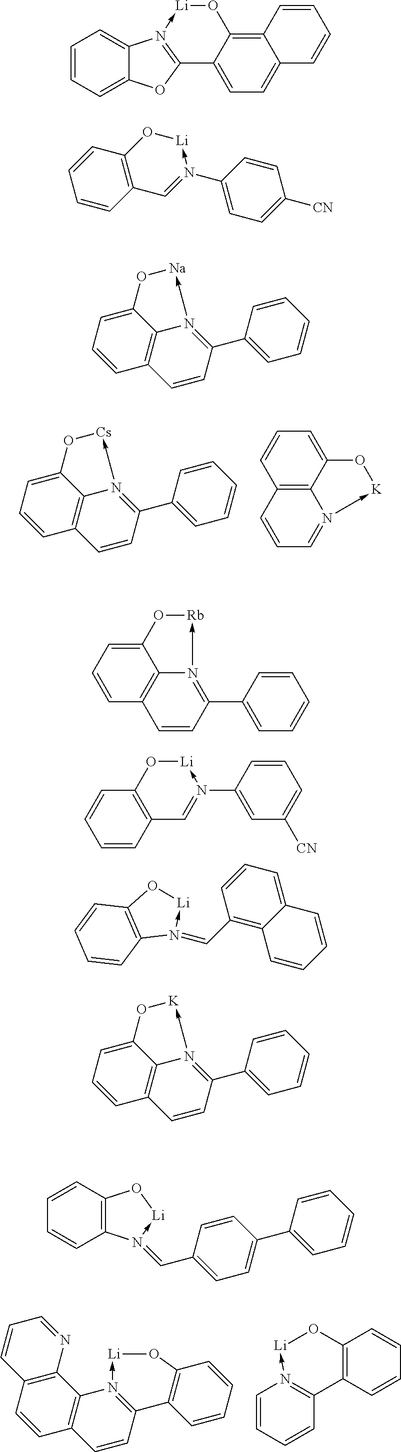

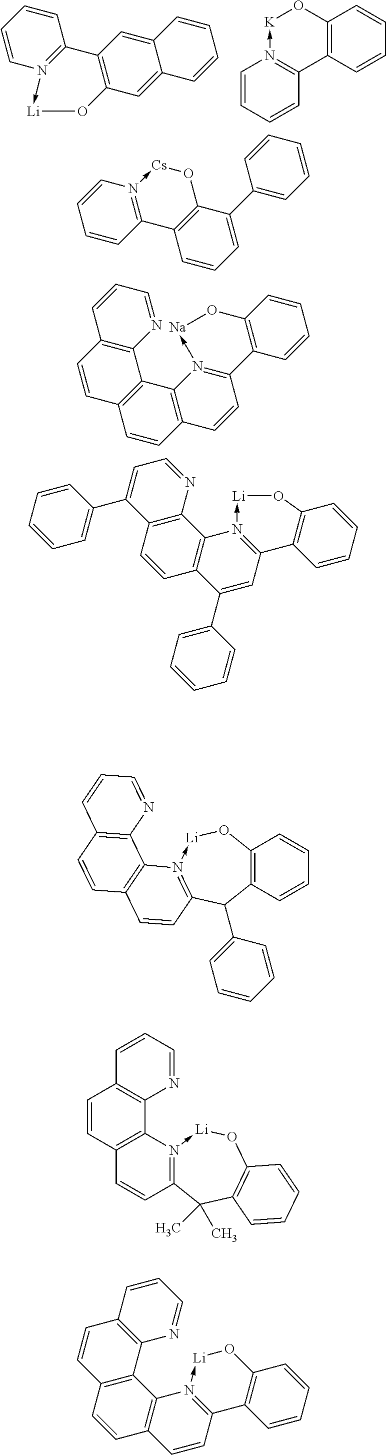

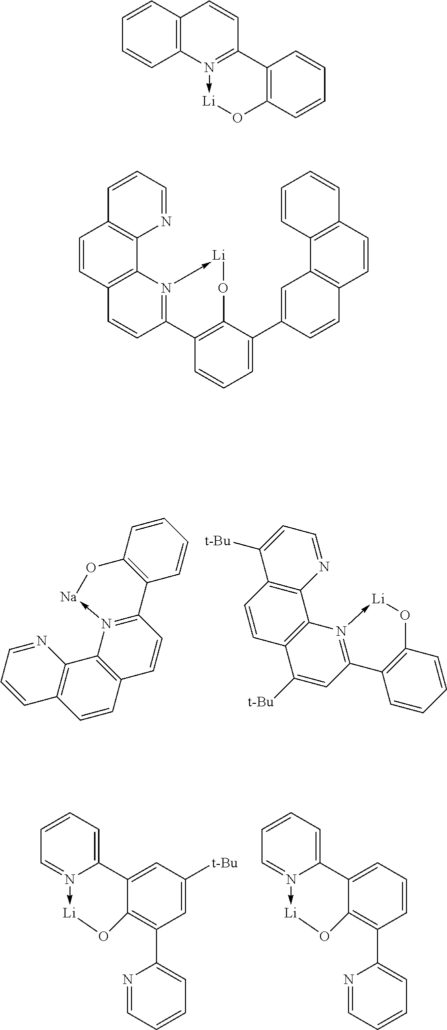

Particularly applicable complexes among the above-mentioned organic alkali metal complexes are the phenanthroline derivatives.

In addition, other examples of n-type dopants include Cr₂hpp₄ (hpp: 1,5,7-triazabicyclo[4.4.0]dec-5-ene), Fe₂hpp₄, Mn₂hpp, Co₂hpp₄, Mo₂hpp₄, W₂hpp₄, Ni₂hpp₄, Cu₂hpp₄, Zn₂hpp₄, and W(hpp)₄. Furthermore, mixtures of organic dopants, such as 4,4′,5,5′-tetracyclohexyl-1,1′,2,2′,3,3′-hexamethyl-2,2′,3,3-tetrahydro-1H,1′H-2,2′-biimidazole, 2,2′-diisopropyl-1,1′,3,3′-tetramethyl-2,2′,3,3′,4,4′,5,5′,6,6′,7,7′-dodeca-1H,1′H-2,2′-dibenzo[d]imidazole, 2,2′-diisopropyl-4,4′,5,5′-tetra(4-methoxyphenyl)-1,1′,3,3′-tetramethyl-2,2′,3,3′-tetrahydro-1H,1′H-2,2′-biimidazole, 2,2′-diisopropyl-4,5-bis(2-methoxyphenyl)-4′,5′-bis(4-methoxyphenyl)-1,1′,3,3′-tetramethyl-2,2′,3,3′-tetrahydro-1H,1′H-2,2′-biimidazole, or 2,2′-diisopropyl-4,5-bis(2-methoxyphenyl)-4′,5′-bis(3-methoxyphenyl)-1,1′,3,3′-tetramethyl-2,2′,3,3′-tetrahydro-1H,1′H-2,2′-biimidazole, can be used as n-type dopants.

In the electronic device as described herein, the n-type doped organic layer comprises an organic compound as described herein.

In some embodiments, the n-type CGL may further comprise a non-charged phenanthroline derivative. It is known that such phenanthroline derivatives can be used in the electron-transport layers. Available non-charged phenanthropoietin derivatives are 4,7-diphenyl-1,10-phenanthroline, which is also known as diazaphenanthrene or Bphen.

The p-type CGL comprises an electron acceptor or a p-dopant, and in particular comprises a strong electron acceptor. These may comprise inorganic compounds such as metal oxides, metal nitrides, metal carbides, complexes of metal ions with organic ligands, and complexes of transition metal ions with organic ligands. Materials suitable for the p-type CGL may also contain plasma-deposited fluorocarbon polymers (CFx) as described in U.S. Pat. No. 6,208,075, strong electron acceptors (e.g., hexaazabenzophenanthrene derivatives) as described in U.S. Pat. No. 6,720,573B2 and U.S. Pat. App. No. 2004113547A1, or other materials which may be organic electron acceptors (e.g., FJCNQ) or inorganic electron acceptors (e.g., MoO₃, FeCl, or FeF). Liao et al., also described other available electron acceptors those were named as electron accepting materials in U.S. Pat. No. 11,867,707, which is incorporated herein by reference.

In some embodiments, the p-type CGL may be a monolayer made of electron accepting materials.

In some embodiments, the p-type CGL may be made of hole-transport materials as a host, and doped with p-type organic dopants, i.e., p-dopants.

Electron acceptors (p-dopants) such as tetracyanoquinodimethane (TCNQ) or 2,3,5,6-tetrafluorotetracyano-1,4-benzoquinodimethane (F4TCNQ) are well known. Reference can be made to the articles M. Pfeiffer, A. Beyer, T. Fritz, K. Leo, App. Phys. Lett., 73(22), 3202-3204 (1998) and J. Blochwitz, M. Pfeiffer, T. Fritz, K. Leo, App. Phys. Lett., 73(6), 729-731 (1998). Due to the defects (low molecular weight and strong volatility) of TCNQ and F4TCNQ in specific applications, some preferred strong electron acceptors (disclosed in DE102013205093A1, WO2009003455A1, CN101330129B, etc.) can be selected from the following:

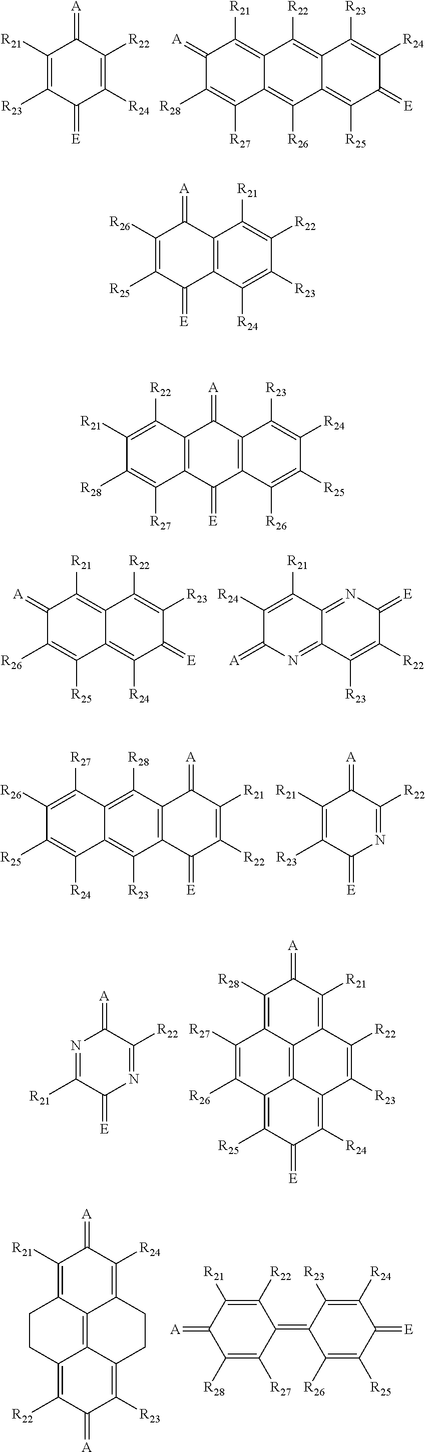

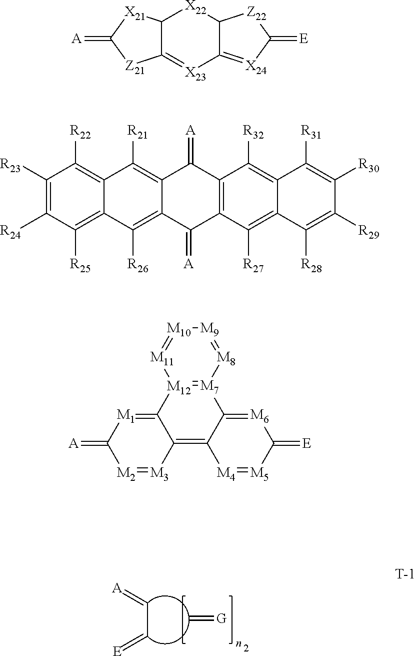

Where each of A, E, and G is independently selected from O, S, SO₂, C(R₂₁R₂₂), or N(R₂₁); each of R₂₁ to R₂₈ at each occurrence is independently selected from H, D, F, Cl, CN, NO₂, CF₃, a perfluoroalkyl group, a sulfone group, a cross-linkable group, a substituted/unsubstituted aromatic or heteroaromatic ring system containing 5 to 40 ring atoms, an aryloxy or heteroaryloxy group containing 5 to 40 ring atoms, or any combination thereof, where one or more R₂₁—R₂₈ may form a monocyclic or polycyclic aliphatic or aromatic ring system with each other and/or with rings bonded thereto; each of M₁ to M₁₂ is N or CR₂₉, each R₂₉ is identically defined as R₂₁-R₂₈; each of X₂₁ to X₂₄ is CR or N; when each of X₂₁ to X₂₄ is independently selected from CR, each R may be the same or different, but at least one thereof comprises at least one electron-withdrawing group; each of Z₂₁ and Z₂₂ is independently selected from O, S, Se, S═O, or SO₂; n₂ in formula T-1 is an integer from 1 to 4.

Some examples of electron acceptors are disclosed in patents such as TW200629362A, EP2690662A, CN101346830A, DE102013205093A, CN109912619A, etc., and specific examples of electron acceptors (p-dopants) are selected from the following:

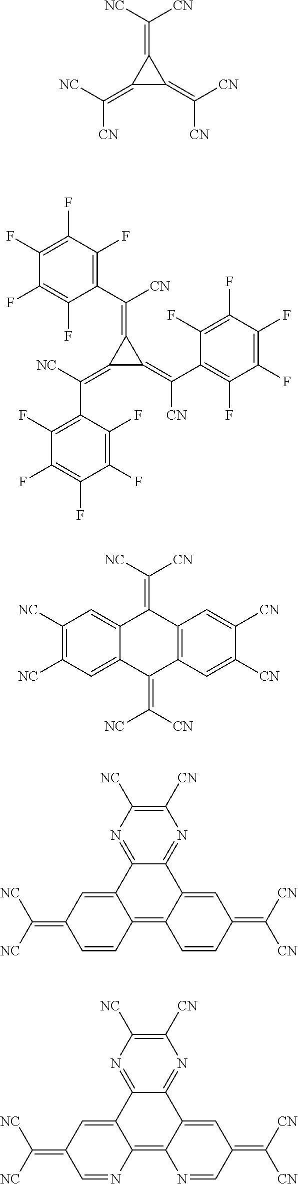 [SMILES: N#CC(C#N)=C1C(=C(C#N)C#N)C1=C(C#N)C#N ; N#CC(=C1C(=C(C#N)c2c(F)c(F)c(F)c(F)c2F)C1=C(C#N)c1c(F)c(F)c(F)c(F)c1F)c1c(F)c(F)c(F)c(F)c1F ; N#CC(C#N)=c1c2cc(C#N)c(C#N)cc2c(=C(C#N)C#N)c2cc(C#N)c(C#N)cc12 ; N#CC(C#N)=c1ccc2c3ccc(=C(C#N)C#N)cc3c3nc(C#N)c(C#N)nc3c2c1 ; N#CC(C#N)=c1cnc2c(c1)c1nc(C#N)c(C#N)nc1c1cc(=C(C#N)C#N)cnc12]

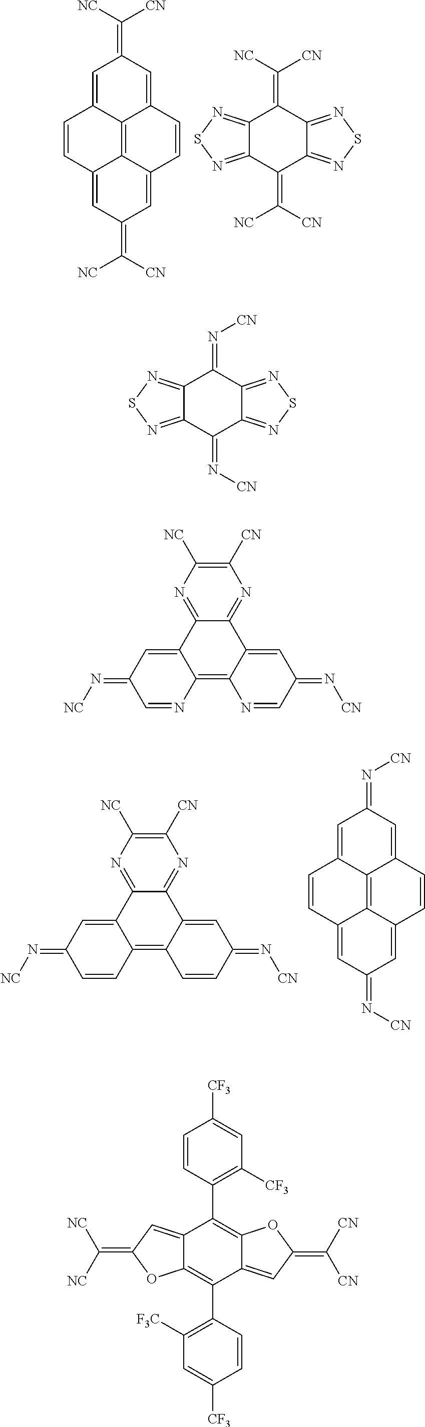

In addition, the organic light-emitting device according to an embodiment of the present disclosure comprises a substrate, an anode, a cathode, and two or more stacked layers disposed between the cathode and the anode, where each of the two or more stacked layers independently comprises a light-emitting layer, a CGL is disposed between the two or more stacked layers, and the CGL comprises an organic compound represented by formula (I).

Furthermore, the organic light-emitting device according to an embodiment of the present disclosure comprises an anode, the first stacked layer comprising the first light-emitting layer disposed on the anode, a CGL disposed on the first stacked layer, a second stacked layer comprising the second light-emitting layer disposed on the CGL, and a cathode disposed on the second stacked layer. As used herein, the CGL is preferably an n-type CGL comprising an organic compound represented by formula (I). In addition, the first stacked layer and the second stacked layer may each independently further comprise one or more above-mentioned layers consisting of a hole-injection layer, a hole-transport layer, a hole-blocking layer, an electron-transport layer, an electron-injection layer, etc.

In addition to the organic compound represented by formula (I), the CGL may also contain the dopants known in the art.

The substrate should be opaque or transparent. A transparent substrate could be used to produce a transparent light-emitting device (for example: Bulovic et al., Nature, 1996, 380, p29, and Gu et al., Appl. Phys. Lett., 1996, 68, p2606). The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg) >150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level/valence band energy level of the emitter of the light-emitting layer or the p-type semiconductor materials of the hole-injection layer (HIL)/hole-transport layer (HTL)/electron-blocking layer (EBL)<0.5 eV, preferably <0.3 eV, and most preferably <0.2 eV Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level/conduction band energy level of the emitter of the light-emitting layer, or the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1. The entire contents of these three documents are hereby incorporated herein for reference.

Where examples of EIM/ETM materials are not particularly limited, and any metal complex or organic compound may be used as EIM/ETM as long as they can transfer electrons. Preferred organic EIM/ETM materials may be selected from the group consisting of tris(8-quinolinolato) aluminum (AlQ3), phenazine, phenanthroline, anthracene, phenanthrene, fluorene, bifluorene, spiro-bifluorene, p-phenylacetylene, pyridazine, pyrazine, triazine, triazole, imidazole, quinoline, isoquinoline, quinazoline, oxazole, isoxazole, oxadiazole, thiadiazole, pyridine, pyrazole, pyrrole, pyrimidine, acridine, pyrene, perylene, trans-indenofluorene, cis-indenonfluorene, dibenzol-indenofluorene, indenonaphthalene, benzanthracene, azaphosphole, azaborole, aromatic ketones, lactams, and derivatives thereof.

The hole-blocking layer (HBL) is typically used to block holes from the adjacent functional layers, particularly the light-emitting layers. In contrast to a light-emitting device without a blocking layer, the presence of HBL usually results in an increase in luminous efficiency. The hole-blocking material (HBM) of the hole-blocking layer (HBL) requires a lower HOMO than the adjacent functional layer, such as the light-emitting layer. In some embodiments, the HBM has a greater energy level of excited state than the adjacent light-emitting layer, such as a singlet or triplet energy level, depending on the emitter. In some embodiments, the HBM has an electron-transport function. Typically, EIM/ETM materials with deep HOMO levels may be used as HBM.

In one aspect, the compounds those may be used as EIM/ETM/HBM may be the molecules comprising one of the following groups.

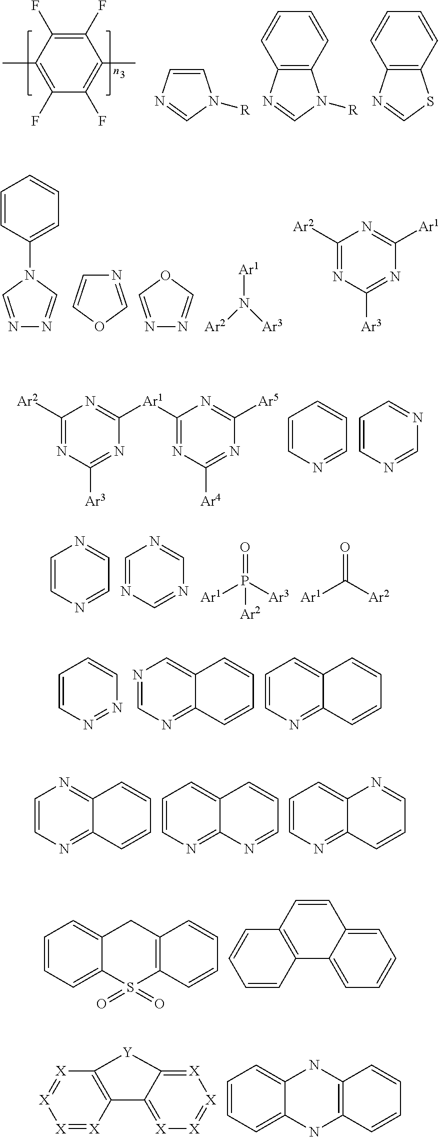

Y at each occurrence is independently selected from C(R)₂, NR, O, S; X at each occurrence is CR or N; each of Ar¹ to Ar⁵ at each occurrence is an aromatic group or a heteroaromatic group; R at each occurrence is independently selected from the following groups: H, deuterium, halogen atoms (F, Cl, Br, I), cyano, alkyl, alkoxy, amino, alkenyl, alkynyl, aralkyl, heteroalkyl, aryl, and heteroaryl; n₃ is an integer from 1 to 20.

In another aspect, examples of metal complexes those may be used as EIM/ETM may include, but not limited to, the following general structures:

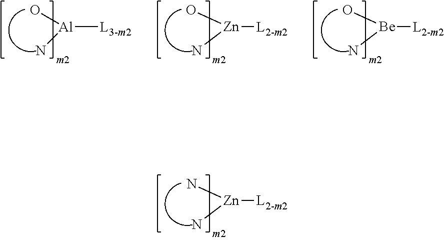

(O—N) or (N—N) is a bidentate ligand, where the metal coordinates with O, N, or N, N; L is an auxiliary ligand; m2 is an integer with the value from 1 to the maximum coordination number of this metal.

Suitable examples of EIM/ETM compounds are listed below:

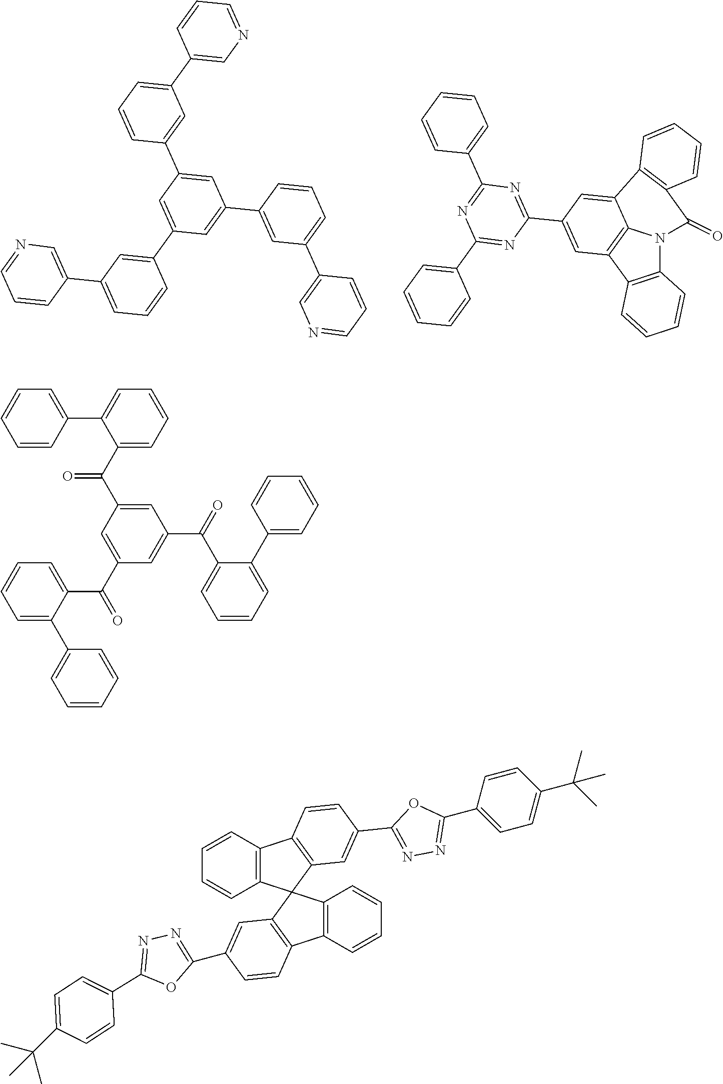

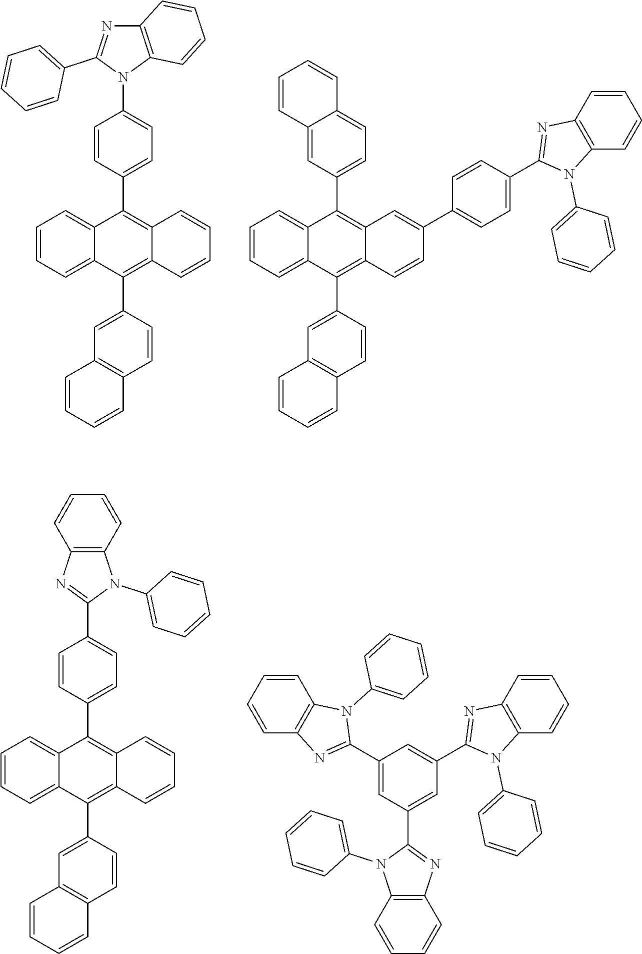

In some embodiments, the above-mentioned n-type dopant may be used as an EIM.

In manufacturing the organic electronic devices, the organic compound may be formed into a functional layer by printing or coating methods, and vacuum deposition. Where suitable printing or coating techniques include, but not limited to, gravure printing, ink-jet printing, nozzle printing, typographic printing, screen printing, dip coating, spin coating, blade coating, roller printing, torsion roll printing, planographic printing, flexographic printing, rotary printing, spray coating, brush coating, pad printing, slit die coating, etc. Preferred techniques are gravure printing, nozzle printing, and ink-jet printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, viscosity, etc.

The present disclosure further provides the applications of organic electronic devices in various electronic equipment, including, but not limited to, display devices, lighting equipment, light sources, sensors, etc.

In another aspect, the present disclosure also provides a compound comprising a structure of formula (1):

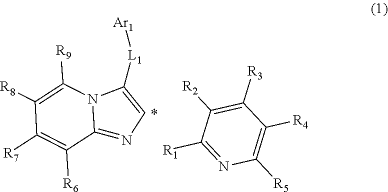

Where any one of R₁-R₅ is directly linked to * via a single bond; R₁-R₉ are identically defined as described herein; L₁ is a substituted/unsubstituted aromatic group containing 6 to 30 ring atoms; Ar₁ is selected from a substituted/unsubstituted aromatic or heteroaromatic group containing 5 to 40 ring atoms, an aryloxy or heteroaryloxy group containing 5 to 40 ring atoms, or any combination thereof, where Ar₁ may form a monocyclic or polycyclic aliphatic or aromatic ring systems with rings bonded thereto, and the ring atom of Ar₁ is directly linked to the ring atom of L₁ via a single bond.

In some embodiments, the compound of formula (1) can be used as an electron-injection material (EIM), an electron-transport material (ETM), a hole-blocking material (HBM), or a CGL material.

As the EIM or ETM, the compound should have an appropriate LUMO energy level. In some embodiments, the LUMO of the compound according to formula (1) ≤−2.7 eV, preferably ≤−2.75 eV, more preferably ≤−2.8 eV, further preferably ≤−2.85 eV, and most preferably ≤−2.9 eV

As the HBM, the compound should have an appropriate HOMO energy level. In some embodiments, the HOMO of the compound according to formula (1) ≤−5.7 eV, preferably ≤−5.8 eV, and most preferably ≤−5.9 eV.

As an organic functional material, it is desirable to have good thermal stability. Generally, the glass transition temperature (Tg) of the compound according to formula (1) ≥100° C., preferably ≥140° C., more preferably ≥180° C.

In some embodiments, the (HOMO-(HOMO-1)) of the compound according to formula (1) ≥0.2 eV, preferably ≥0.3 eV, more preferably ≥0.4 eV, and most preferably ≥0.45 eV

In some embodiments, the ((LUMO+1)-LUMO) of the compound according to formula (1) ≥0.1 eV, preferably ≥0.15 eV, more preferably ≥0.18 eV, and most preferably ≥0.2 eV

Other embodiments of the compound according to formula (1) are included in the above description and are not be repeated.

In yet another aspect, the present disclosure further provides a polymer comprising at least one repeating unit, where the at least one repeating unit is selected from at least one structure of formula (I) or formula (1).

In yet another aspect, the present disclosure further provides a formulation comprising an organic solvent, at least one compound of formula (I) or formula (1) as described herein, or at least one polymer as described herein.

In yet another aspect, the present disclosure further provides a mixture comprising a compound of formula (I) or formula (1) as described herein, or a polymer as described herein, and at least one organic functional material, where the at least one organic functional material is selected from a hole-injection material, a hole-transport material, an electron-transport material, an electron-injection material, an electron-blocking material, a hole-blocking material, an emitting material (including a fluorescent emitting material, a phosphorescent emitting material, a thermally activated delayed fluorescence (TADF) material), or a host material.

In yet another aspect, the present disclosure further provides an organic electronic device comprising at least one compound of formula (1) or at least one polymer or at least one mixture as described herein. Generally, the organic electronic device comprises a cathode, an anode, and a functional layer disposed between the cathode and the anode, wherein the functional layer comprises at least one compound or at least one polymer or at least one mixture as described herein.

The organic electronic device may be selected from, but not limited to, a color converter, an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, an organic plasmon emitting diode (OPED), etc, particularly preferably an organic electroluminescent device, such as an OLED, an OLEEC, an organic light emitting field effect transistor.

The OLED comprises a substrate, an anode, at least one functional layer, and a cathode. The at least one functional layer comprises a compound of formula (1) or a polymer or a mixture as described herein, or is prepared using the formulation as described herein. The at least one functional layer is selected from a hole-injection layer (HIL), a hole-transport layer (HTL), an emitting layer (EML), an electron-blocking layer (EBL), an electron-injection layer (EIL), an electron-transport layer (ETL), a hole-blocking layer (HBL), a charge generation layer (CGL); preferably, the at least one functional layer is an electron-transport layer or a charge generation layer.

The emitting wavelength of the OLED is between 300 nm and 1200 nm, preferably between 350 nm and 1000 nm, more preferably between 400 nm and 900 nm.

It is an object of the present disclosure to provide a material for the evaporation-based OLEDs. For this purpose, the molecular weight of the compound according to formula (1) 1000 g/mol, preferably 900 g/mol, more preferably 850 g/mol, further preferably 800 g/mol, and most preferably 700 g/mol.

Another object of the present disclosure is to provide a material for the printed OLEDs. For this purpose, the molecular weight of the compound according to formula (1) ≥700 g/mol, preferably ≥800 g/mol, more preferably ≥900 g/mol, further preferably ≥1000 g/mol, and most preferably ≥1100 g/mol.

In some embodiments, the glass transition temperature (Tg) of the compound according to formula (1) ≥100° C. In some embodiments, Tg ≥120° C. In some embodiments, Tg ≥140° C. In some embodiments, Tg ≥160° C. In some embodiments, Tg ≥180° C.

In some embodiments, the compound of formula (1) is partially deuterated; preferably 10% or more of total H, more preferably 20% or more of total H, further preferably 30% or more of total H, and most preferably 40% or more of total H, are deuterated.

In some embodiments, the compound of formula (1) has a solubility of ≥10 mg/mL in toluene at 25° C., preferably ≥15 mg/mL, and most preferably ≥20 mg/mL.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### SPECIFIC EMBODIMENT

### Synthesis of Compounds

### Synthesis of Intermediate 1

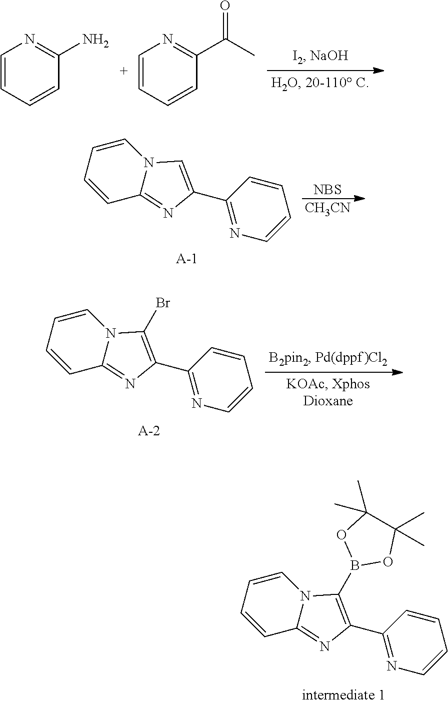

2-aminopyridine (109.4 g, 1.15 mol), 2-acetylpyridine (60.6 g, 0.5 mol), and iodine (152.3 g, 0.6 mol) were added to a 1 L single-necked flask, and the resulting mixture was reacted at 110° C. for 4 h, then at 70° C. for another 12 h. After adding sodium hydroxide (200 g, 5 mol) and 200 mL of water, the resulting solution was heated to 100° C. and reacted for 1 h. After the reaction was completed, the resulting solution was diluted with 200 mL of dichloromethane, and 6M HCl solution was added until PH=8. After the extraction with 300 mL of dichloromethane, the resulting organic phase was washed with water, dried over anhydrous Na₂SO₄, filtered, and the excess solvent was removed under reduced pressure distillation, then the resulting sample was further purified by silica gel column chromatography (eluent:petroleum ether:ethyl acetate=1: 3) to yield about 48.8 g (yield: about 50%) of intermediate A-1. MS (ASAP)=195.2.

Intermediate A-1 (39.0 g, 0.2 mol) was added to a 500 mL three-necked flask, then dissolved in 350 mL of acetonitrile. N-bromosuccinimide (42.7 g, 0.24 mol) was added in portions into the above solution, and the resultant mixture was reacted at 30° C. for 5 h in dark. After the reaction was completed, 20 mL of water was added, the result was extracted with 300 mL of dichloromethane. After that, the resulting organic phases were dried over anhydrous Na₂SO₄, filtered, and the excess solvent was removed under reduced pressure distillation, then the resulting sample was further purified by silica gel column chromatography (eluent: ethyl acetate) to yield about 53.7 g (yield: about 98%) of intermediate A-2. MS (ASAP)=274.1.

Intermediate A-2 (41.1 g, 0.15 mol), bis(pinacolato)diboron (41.9 g, 0.165 mol), dichloro[1,1′-bis(diphenylphosphino)ferrocene]palladium (5.9 g, 8 mmol), potassium acetate (32.4 g, 0.33 mol), and 2-dicyclohexylphosphino-2′,4′,6′-triisopropylbiphenyl (15.7 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent:petroleum ether:ethyl acetate=1:3) to yield about 29.4 g (yield: about 61%) of intermediate 1. MS (ASAP)=321.2.

### Synthesis of Compound 1

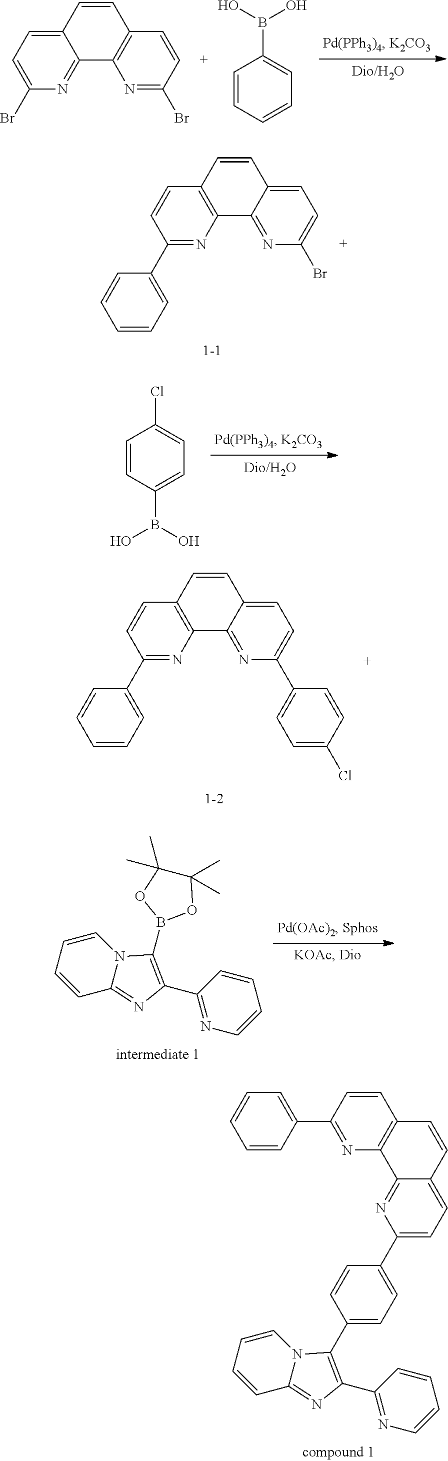

2,9-dibromo-1,10-phenanthroline (84.5 g, 0.25 mol), phenylboronic acid (30.5 g, 0.25 mol), tetrakis(triphenylphosphine)palladium (8.7 g, 7.5 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted for 8 h. After cooling down to room temperature, 200 mL of water was added, then the resulting mixture was extracted with ethyl acetate (250 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 68.7 g (yield: about 82%) of intermediate 1-1. MS (ASAP)=335.2.

Intermediate 1-1 (67.0 g, 0.2 mol), 4-chlorophenylboronic acid (32.8 g, 0.21 mol), tetrakis(triphenylphosphine)palladium (6.9 g, 6 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted overnight. After cooling down to room temperature, 200 mL of water was added, then the resulting mixture was extracted with ethyl acetate (250 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 61.6 g (yield: about 84%) of intermediate 1-2. MS (ASAP)=366.9.

Intermediates 1-2 (55.0 g, 0.15 mol), intermediate 1 (53.0 g, 0.165 mol), palladium acetate (2.0 g, 8 mmol), potassium acetate (32.4 g, 0.33 mol), and 2-dicyclohexylphosphino-2′,6′-dimethoxybiphenyl (13.5 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 52.8 g (yield: about 67%) of compound 1. MS (ASAP)=525.6.

### Synthesis of Compound 2

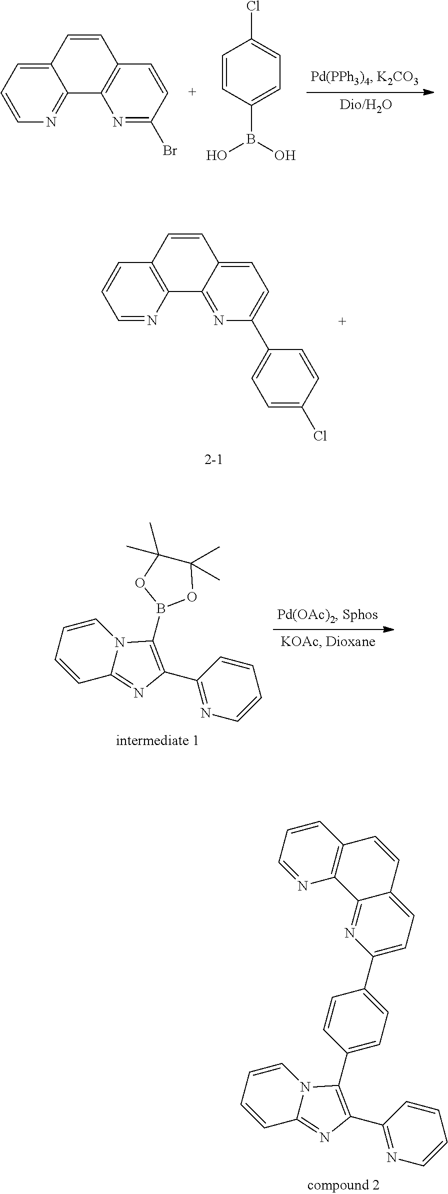 [SMILES: Brc1ccc2ccc3cccnc3c2n1 ; OB(O)c1ccc(Cl)cc1 ; Clc1ccc(-c2ccc3ccc4cccnc4c3n2)cc1 ; CC1(C)OB(c2c(-c3ccccn3)nc3ccccn23)OC1(C)C ; c1ccc(-c2nc3ccccn3c2-c2ccc(-c3ccc4ccc5cccnc5c4n3)cc2)nc1]

2-Bromo-1,10-phenanthroline (51.8 g, 0.2 mol), 4-chlorophenylboronic acid (32.8 g, 0.21 mol), tetrakis(triphenylphosphine)palladium (6.9 g, 6 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted overnight. After cooling down to room temperature, 200 mL of water was added, then the resulting mixture was extracted with ethyl acetate (250 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 51.8 g (yield: about 89%) of intermediate 2-1. MS (ASAP)=290.8.

Intermediate 2-1 (43.6 g, 0.15 mol), intermediate 1 (53.0 g, 0.165 mol), palladium acetate (2.0 g, 8 mmol), potassium acetate (32.4 g, 0.33 mol), and 2-dicyclohexylphosphino-2′,6′-dimethoxybiphenyl (13.5 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 49.9 g (yield: about 74%) of compound 2. MS (ASAP)=449.5.

### Synthesis of Compound 3

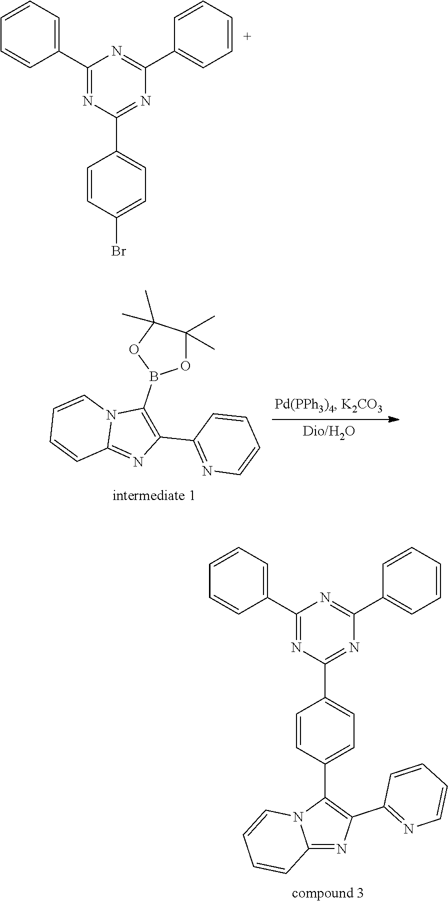 [SMILES: Brc1ccc(-c2nc(-c3ccccc3)nc(-c3ccccc3)n2)cc1 ; CC1(C)OB(c2c(-c3ccccn3)nc3ccccn23)OC1(C)C ; c1ccc(-c2nc(-c3ccccc3)nc(-c3ccc(-c4c(-c5ccccn5)nc5ccccn45)cc3)n2)cc1]

2-(4-Bromophenyl)-4,6-diphenyl-1,3,5-triazine (97.1 g, 0.25 mol), intermediate 1 (84.3 g, 0.263 mol), tetrakis(triphenylphosphine)palladium (14.4 g, 12.5 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted for 8 h. After cooling down to room temperature, 200 mL of water was added, then the resulting mixture was extracted with ethyl acetate (500 mL*3). The organic phases were then combined, dried over anhydrous Na₂SO₄, the excess solvent was removed under reduced pressure distillation, then the residue was recrystallized with o-xylene to yield 80.4 g (yield: about 64%) of compound 3. MS (ASAP)=502.6.

### Synthesis of Compound 4

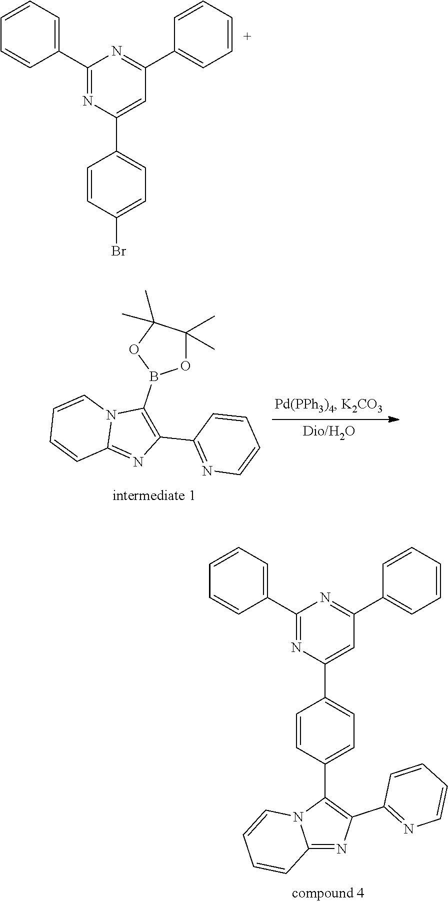 [SMILES: Brc1ccc(-c2cc(-c3ccccc3)nc(-c3ccccc3)n2)cc1 ; CC1(C)OB(c2c(-c3ccccn3)nc3ccccn23)OC1(C)C ; c1ccc(-c2cc(-c3ccc(-c4c(-c5ccccn5)nc5ccccn45)cc3)nc(-c3ccccc3)n2)cc1]

4-(4-Bromophenyl)-2,6-diphenylpyrimidine (96.8 g, 0.25 mol), intermediate 1 (84.3 g, 0.263 mol), tetrakis(triphenylphosphine)palladium (14.4 g, 12.5 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted for 8 h. After cooling down to room temperature, 200 mL of water was added, then the resulting mixture was extracted with ethyl acetate (500 mL*3). The organic phases were then combined, dried over anhydrous Na₂SO₄, the excess solvent was removed under reduced pressure distillation, then the residue was recrystallized with o-xylene to yield 87.8 g (yield: about 70%) of compound 4. MS (ASAP)=501.6.

### Synthesis of Compound 5

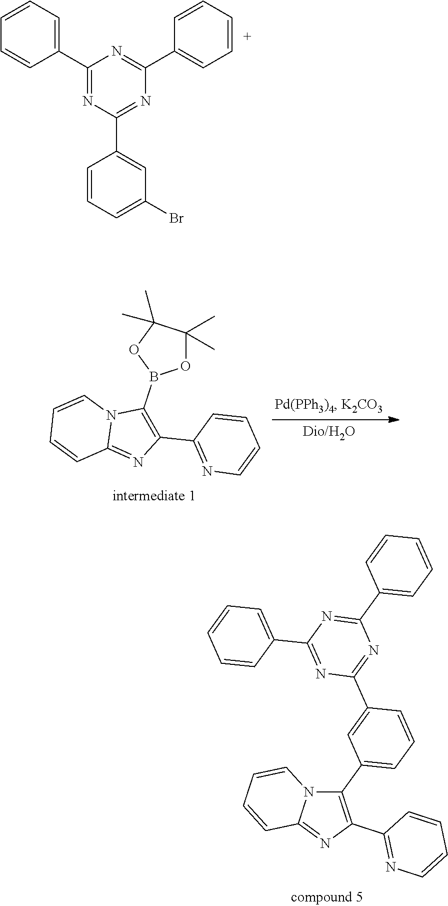 [SMILES: Brc1cccc(-c2nc(-c3ccccc3)nc(-c3ccccc3)n2)c1 ; CC1(C)OB(c2c(-c3ccccn3)nc3ccccn23)OC1(C)C ; c1ccc(-c2nc(-c3ccccc3)nc(-c3cccc(-c4c(-c5ccccn5)nc5ccccn45)c3)n2)cc1]

2-(3-Bromophenyl)-4,6-diphenyl-1,3,5-triazine (97.1 g, 0.25 mol), intermediate 1 (84.3 g, 0.263 mol), tetrakis(triphenylphosphine)palladium (14.4 g, 12.5 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted for 8 h. After cooling down to room temperature, 200 mL of water was added, then the resulting mixture was extracted with ethyl acetate (500 mL*3). The organic phases were then combined, dried over anhydrous Na₂SO₄, the excess solvent was removed under reduced pressure distillation, then the residue was recrystallized with o-xylene to yield 80.4 g (yield: about 64%) of compound 5. MS (ASAP)=502.6.

### Synthesis of Compound 6

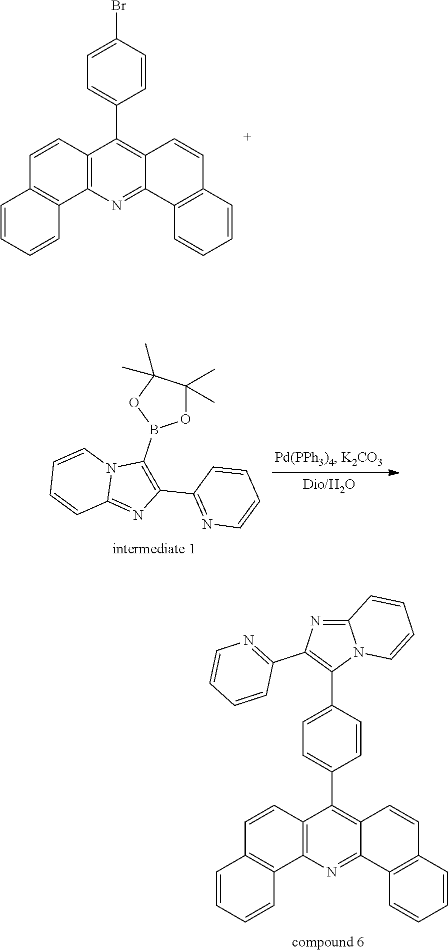 [SMILES: Brc1ccc(-c2c3ccc4ccccc4c3nc3c2ccc2ccccc23)cc1 ; CC1(C)OB(c2c(-c3ccccn3)nc3ccccn23)OC1(C)C ; c1ccc(-c2nc3ccccn3c2-c2ccc(-c3c4ccc5ccccc5c4nc4c3ccc3ccccc34)cc2)nc1]

7-(4-Bromophenyl)dibenzo[C,H]acridine (108.6 g, 0.25 mol), intermediate 1 (84.3 g, 0.263 mol), tetrakis(triphenylphosphine)palladium (14.4 g, 12.5 mmol), and potassium carbonate (69 g, 0.5 mol) were added to a 1 L three-necked flask. After adding 500 mL of 1,4-dioxane and 100 mL of water, the resulting mixture was heated to 100° C. under N₂ atmosphere and reacted for 8 h. After cooling down to room temperature, 200 mL of water was added, then the resulting mixture was extracted with ethyl acetate (500 mL*3). The organic phases were then combined, dried over anhydrous Na₂SO₄, the excess solvent was removed under reduced pressure distillation, then the resulting sample was further purified by silica gel column chromatography (eluent:petroleum ether:ethyl acetate=1:1) to yield about 98.9 g (yield: about 72%) of compound 6. MS (ASAP)=548.7.

### Synthesis of Compound 7

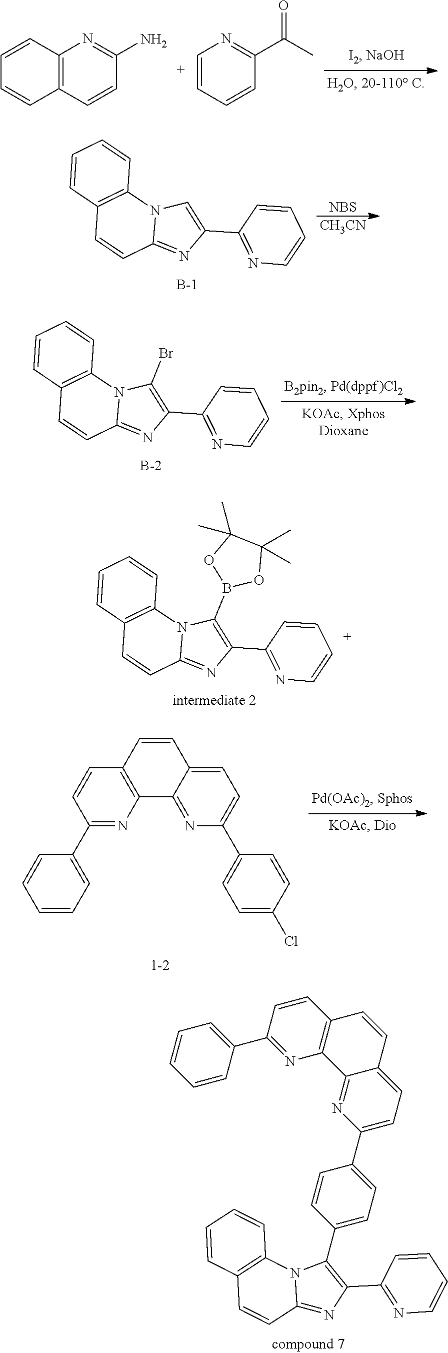

2-Aminoquinoline (165.8 g, 1.15 mol), 2-acetylpyridine (60.6 g, 0.5 mol), and iodine (152.3 g, 0.6 mol) were added to a 1 L single-necked flask, and the resulting mixture was reacted at 110° C. for 4 h, then at 70° C. for another 12 h. After adding sodium hydroxide (200 g, 5 mol) and 200 mL of water, the resulting solution was heated to 100° C. and reacted for 1 h. After the reaction was completed, the resulting solution was diluted with 200 mL of dichloromethane, and 6M HCl solution was added until PH=8. After the extraction with 300 mL of dichloromethane, the resulting organic phase was washed with water, dried over anhydrous Na₂SO₄, filtered, and the excess solvent was removed under reduced pressure distillation, then the resulting sample was further purified by silica gel column chromatography (eluent:petroleum ether:ethyl acetate=1:3) to yield about 68.7 g (yield: about 56%) of intermediate B-1. MS (ASAP)=245.3.

Intermediate B-1 (49.1 g, 0.2 mol) was added to a 500 mL three-necked flask, then dissolved in 350 mL of acetonitrile. N-bromosuccinimide (42.7 g, 0.24 mol) was added in portions into the above solution, and the resultant mixture was reacted at 30° C. for 5 h in dark. After the reaction was completed, 20 mL of water was added, the result was extracted with 300 mL of dichloromethane. After that, the resulting organic phases were dried over anhydrous Na₂SO₄, filtered, and the excess solvent was removed under reduced pressure distillation, then the resulting sample was further purified by silica gel column chromatography (eluent: ethyl acetate) to yield about 64.8 g (yield: about 100%) of intermediate B-2. MS (ASAP)=324.2.

Intermediate B-2 (41.1 g, 0.15 mol), bis(pinacolato)diboron (41.9 g, 0.165 mol), dichloro[1,1′-bis(diphenylphosphino)ferrocene]palladium (5.9 g, 8 mmol), potassium acetate (32.4 g, 0.33 mol), and 2-dicyclohexylphosphino-2′,4′,6′-triisopropylbiphenyl (15.7 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent:petroleum ether:ethyl acetate=1:3) to yield about 36.2 g (yield: about 65%) of intermediate 2. MS (ASAP)=371.3.

Intermediates 1-2 (55.0 g, 0.15 mol), intermediate 2 (61.3 g, 0.165 mol), palladium acetate (2.0 g, 8 mmol), potassium acetate (32.4 g, 0.33 mol), and 2-dicyclohexylphosphino-2′,6′-dimethoxybiphenyl (13.5 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 62.2 g (yield: about 72%) of compound 7. MS (ASAP)=575.7.

### Synthesis of Compound 8

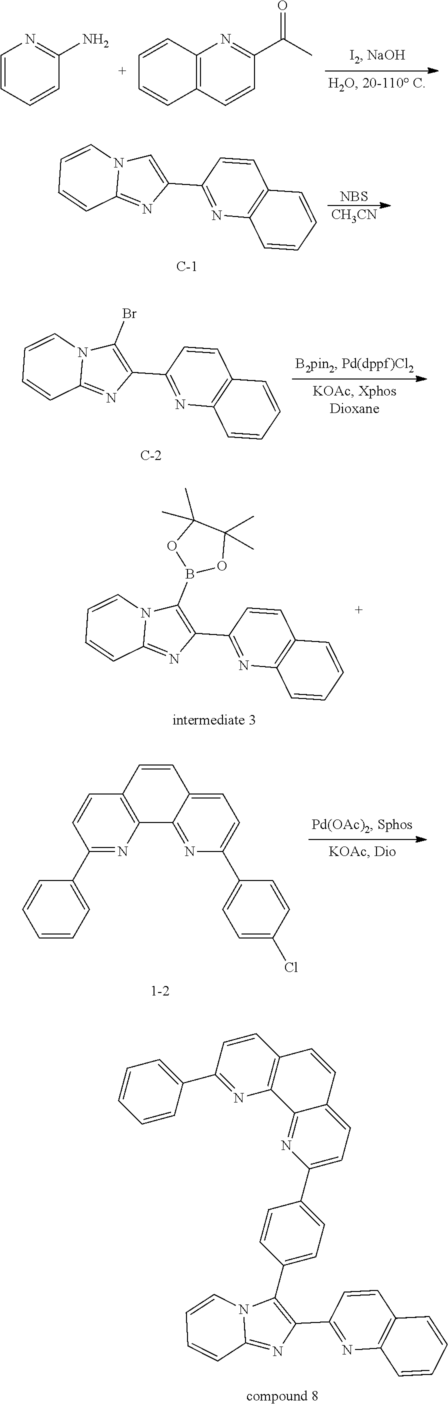

2-Aminopyridine (109.4 g, 1.15 mol), 2-acetylquinoline (85.6 g, 0.5 mol), and iodine (152.3 g, 0.6 mol) were added to a 1 L single-necked flask, and the resulting mixture was reacted at 110° C. for 4 h, then at 70° C. for another 12 h. After adding sodium hydroxide (200 g, 5 mol) and 200 mL of water, the resulting solution was heated to 100° C. and reacted for 1 h. After the reaction was completed, the resulting solution was diluted with 200 mL of dichloromethane, and 6M HCl solution was added until PH=8. After the extraction with 300 mL of dichloromethane, the resulting organic phase was washed with water, dried over anhydrous Na₂SO₄, filtered, and the excess solvent was removed under reduced pressure distillation, then the resulting sample was further purified by silica gel column chromatography (eluent:petroleum ether:ethyl acetate=1:3) to yield about 62.6 g (yield: about 51%) of intermediate C-1. MS (ASAP)=245.3.

Intermediate C-1 (49.1 g, 0.2 mol) was added to a 500 mL three-necked flask, then dissolved in 350 mL of acetonitrile. N-bromosuccinimide (42.7 g, 0.24 mol) was added in portions into the above solution, and the resultant mixture was reacted at 30° C. for 5 h in dark. After the reaction was completed, 20 mL of water was added, the result was extracted with 300 mL of dichloromethane. After that, the resulting organic phases were dried over anhydrous Na₂SO₄, filtered, and the excess solvent was removed under reduced pressure distillation, then the resulting sample was further purified by silica gel column chromatography (eluent: ethyl acetate) to yield about 64.8 g (yield: about 100%) of intermediate C-2. MS (ASAP)=324.2.

Intermediate C-2 (41.1 g, 0.15 mol), bis(pinacolato)diboron (41.9 g, 0.165 mol), dichloro[1,1′-bis(diphenylphosphino)ferrocene]palladium (5.9 g, 8 mmol), potassium acetate (32.4 g, 0.33 mol), and 2-dicyclohexylphosphino-2′,4′,6′-triisopropylbiphenyl (15.7 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent:petroleum ether:ethyl acetate=1:3) to yield about 37.9 g (yield: about 68%) of intermediate 3. MS (ASAP)=371.3.

Intermediates 1-2 (55.0 g, 0.15 mol), intermediate 3 (61.3 g, 0.165 mol), palladium acetate (2.0 g, 8 mmol), potassium acetate (32.4 g, 0.33 mol), and 2-dicyclohexylphosphino-2′,6′-dimethoxybiphenyl (13.5 g, 33 mmol) were added to a 500 mL three-necked flask. After adding 300 mL of dioxane, the resulting mixture was heated to 100° C. under N₂ atmosphere and reacted overnight. After cooling down to room temperature, 300 mL of water was added, then the resulting mixture was separated, and the aqueous phase was extracted with dichloromethane (100 mL*3). The organic phases were then combined, dried over anhydrous Na₂SO₄, the excess solvent was removed under reduced pressure distillation, then the resulting sample was further purified by silica gel column chromatography (eluent: dichloromethane) to yield about 55.3 g (yield: about 64%) of compound 8. MS (ASAP)=575.7.

### 2. Energy Structure of Organic Compounds

The energy level of the organic material can be calculated by quantum computation, for example, using TD-DFT (time-dependent density functional theory) by Gaussian 09W (Gaussian Inc.), the specific simulation methods of which can be found in WO2011141110. Firstly, the molecular geometry is optimized by density functional theory “Ground State/DFT/Default Spin/B3LYP” and the basis set “6-31G (d)” (Charge 0/Spin Singlet), then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO energy levels are calculated using the following calibration formula, where S1 and T1 are used directly.

$$HOMO ( eV ) = ( ( HOMO ( G ) \times 27.212 ) - 0.9899 ) / 1.1206 \\ LUMO ( eV ) = ( ( LUMO ( G ) \times 27.212 ) - 2.0041 ) / 1.385$$

Where HOMO(G) and LUMO(G) are the direct calculation results of Gaussian 09W, in units of Hartree. The results are shown in Table 1 below:

TABLE 1

| | HOMO | LUMO | ΔHOMO | ΔLUMO | S1 | T1 |
|---|---|---|---|---|---|---|
| Materials | (eV) | (eV) | (eV) | (eV) | (eV) | (eV) |
| Compound 1 | −5.72 | −2.81 | 0.49 | 0.18 | 3.16 | 2.42 |
| Compound 2 | −5.71 | −2.74 | 0.63 | 0.13 | 3.23 | 2.49 |
| Compound 3 | −5.88 | −2.87 | 0.60 | 0.03 | 3.17 | 2.65 |
| Compound 4 | −5.89 | −2.69 | 0.60 | 0.16 | 3.33 | 2.73 |
| Compound 5 | −5.87 | −2.83 | 0.57 | 0.01 | 3.20 | 2.74 |
| Compound 6 | −5.90 | −2.92 | 0.16 | 0.49 | 3.34 | 2.25 |
| Compound 7 | −5.81 | −2.82 | 0.28 | 0.19 | 3.28 | 2.47 |
| Compound 8 | −5.73 | −2.79 | 0.48 | 0.16 | 3.12 | 2.41 |
| Bphen | −6.36 | −2.56 | 0.33 | 0.02 | 3.56 | 2.69 |

### 3. Preparation and Characterization of OLED Devices

The preparation process of the OLED devices above will be described in detail with reference to specified examples below.

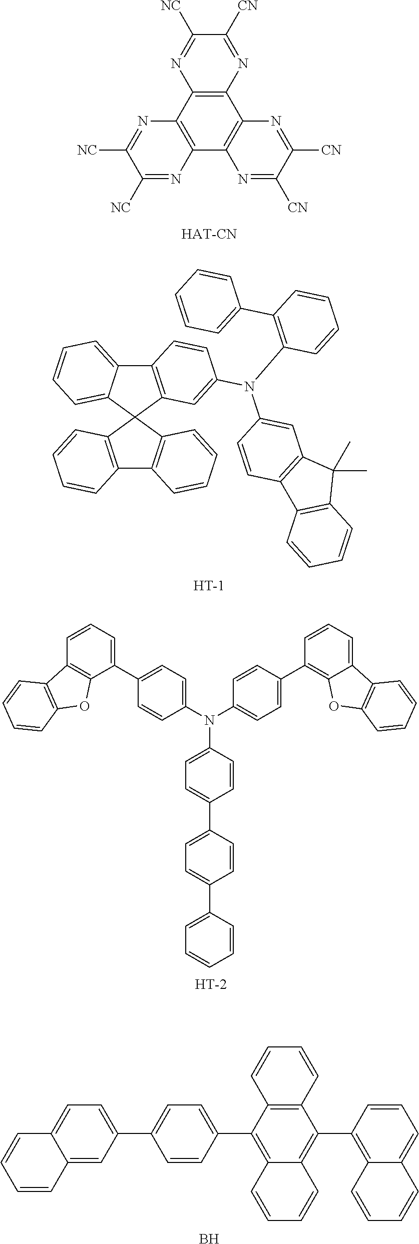

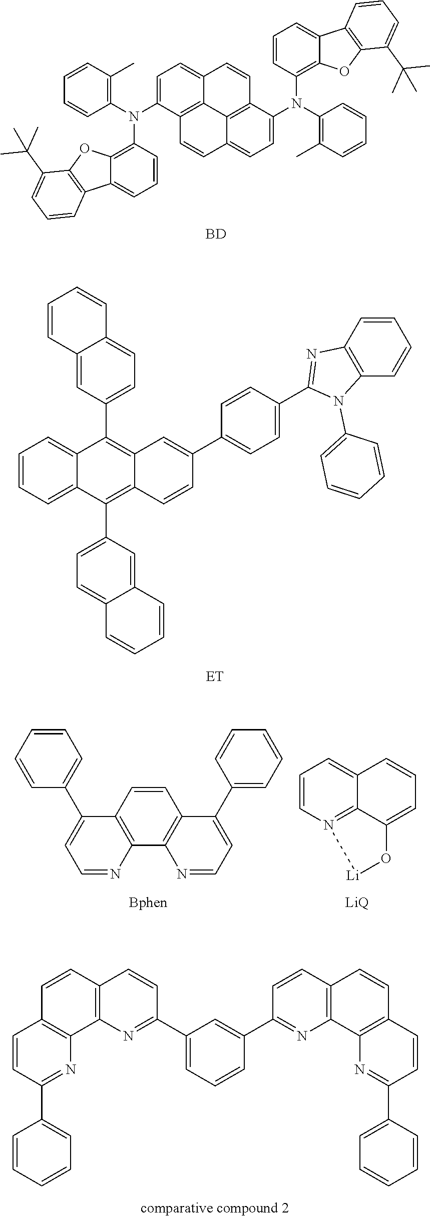

### Device Example 1-Device Example 6

a. Cleaning of the ITO (Indium Tin Oxide) conductive glass substrate: the substrates are washed with various solvents (such as one or more of chloroform, ketone, or isopropyl alcohol), and then treated with UV and ozone.
b. Evaporation: The resultant ITO substrate was mounted on a vacuum deposition apparatus in high vacuum (1×10⁻⁶ mbar), and HAT-CN was then vacuum-deposited on the anode to form a hole-injection layer having a thickness of 30 nm, then two hole-transport layers, HT-1 (50 nm) and HT-2 (10 nm) were deposited on the HIL in vaccum deposition sequentially. Then BH and BD in two different evaportaion sources were deposited at a weight ratio of 100:3 to form a light-emitting layer having a thickness of 25 nm. Subsequently, compound ET and LiQ were placed in two different evaporation units, and co-deposited at a weight ratio of 50:50 to form an electron-transport layer. LiQ was then deposited on the electron-transport layer to form an electron-injection layer having a thickness of 1 nm, and Al was deposited on the electron-injection layer to form a cathode having a thickness of 100 nm.
c. Encapsulation: encapsulating the device in a nitrogen-regulated glove box with UV curable resin.

The OLEDs were fabricated based on the device structure of HI(10)/HT-1(50)/HT-2(10)/BH:BD=100:3(25)/ET:LiQ=50:50(30)/LiQ(1)/Al(100).

As shown in Table 2, the device performance of the device examples and the comparative example were tested; where the drive voltage and the current efficiency were measured at a current density of 10 mA/cm²; T95 refers to the relative value of the time at which the luminance of the device examples decreases to 95% of the initial luminance at a constant current density of 20 mA/cm² compared with that of Comparative Example 1.

TABLE 2

| | Electron-transport | drive | current | |
|---|---|---|---|---|
| | materials | voltage | efficiency | |
| | (ETM) | (V) | (cd/A) | T95 |
| Device Example 1 | compound 1 | 4.10 | 7.6 | 157% |
| Device Example 2 | compound 2 | 4.14 | 7.7 | 176% |
| Device Example 3 | compound 3 | 4.08 | 7.5 | 147% |
| Device Example 4 | compound 4 | 4.15 | 7.6 | 153% |
| Device Example 5 | compound 5 | 4.13 | 7.4 | 137% |
| Device Example 6 | compound 7 | 4.11 | 7.3 | 132% |
| Comparative Example 1 | Bphen | 4.35 | 6.8 | 100% |

The current efficiency and lifetime of Device Example 1-Device Example 6 are obviously improved compared with Comparative Example 1, thereby illustrating that applying the compounds as described herein to OLEDs as electron-transport materials can improve the current efficiency and lifetime of the devices, as well as reducing the driving voltage of the devices.

### Device Example 7-Device Example 8

The cleaned ITO substrate was mounted on a vacuum deposition apparatus in high vacuum (1×10⁻⁶ mbar), and HAT-CN was then vacuum-deposited on the anode to form a hole-injection layer having a thickness of 30 nm, then two hole-transport layers, HT-1 (50 nm) and HT-2 (10 nm) were deposited on the HIL in vaccum deposition sequentially. Then BH and BD in two different evaportaion sources were deposited at a weight ratio of 100:3 to form a light-emitting layer having a thickness of 25 nm. Subsequently, compound ET and LiQ were placed in two different evaporation units, and co-deposited at a weight ratio of 50:50 to form an electron-transport layer. After that, N-type CGL materials (compound 1, compound 2, and comparative compound 2) and the dopant Yb were co-deposited at an evaporation rate ratio of 20:1 on the electron-transport layer to form an N-type CGL having a thickness of 20 nm, then HAT-CN was then evaporated on the N-type CGL to form a P-type CGL having a thickness of 10 nm. Then another hole-injection layer, another hole-transport layer, another light-emitting layer, and another electron-transport layer were sequentially evaporated under the same conditions as described herein. LiQ was then evaporated to form an electron-injection layer having a thickness of 1 nm, and Al was deposited on the electron-injection layer to form a cathode having a thickness of 80 nm.

TABLE 3

| | N-type CGL | V@10 | EQE@10 |
|---|---|---|---|
| | materials | mA/cm² | mA/cm² (%) |
| Device Example 7 | compound 1 | 7.46 | 24.10 |
| Device Example 8 | compound 2 | 7.51 | 21.77 |
| Comparative | comparative | 7.77 | 23.89 |
| Example 2 | compound 2 | | |

As shown in Table 3, in the case of the same materials except for the N-type CGL material, the tandem OLED devices, using the compounds of the present disclosure to form N-CGL, exhibit relatively low drive voltage and high external quantum efficiency (EQE).

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. An organic electronic device, comprising a cathode, an anode, at least two light-emitting layers disposed between the cathode and the anode, and a charge generation layer (CGL) disposed between the adjacent two light-emitting layers, wherein the CGL comprises an organic compound of formula (I): 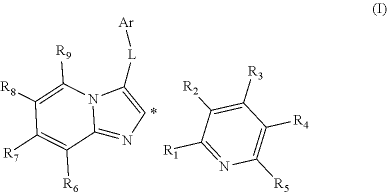
wherein,
any one of R₁-R₅ is directly linked to * via a single bond;
each of R₁ to R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups are capable of being fused to form a ring;
each L is a substituted/unsubstituted aromatic or heteroaromatic group containing 6 to 30 ring atoms;
each Ar is selected from a substituted/unsubstituted aromatic or heteroaromatic group containing 5 to 40 ring atoms, an aryloxy or heteroaryloxy group containing 5 to 40 ring atoms, or any combination thereof, wherein Ars form a monocyclic or polycyclic aliphatic or aromatic ring systems with rings bonded thereto.

2. The organic electronic device according to claim 1, wherein the organic compound comprises a structure of formula (II): 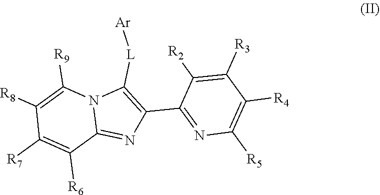
wherein, each of R₂ to R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups are capable of being fused to form a ring; each L is a substituted/unsubstituted aromatic or heteroaromatic group containing 6 to 30 ring atoms; each Ar is selected from a substituted/unsubstituted aromatic or heteroaromatic group containing 5 to 40 ring atoms, an aryloxy or heteroaryloxy group containing 5 to 40 ring atoms, or any combination thereof, wherein Ars form a monocyclic or polycyclic aliphatic or aromatic ring systems with rings bonded thereto.

3. The organic electronic device according to claim 1, wherein the organic compound comprises a structure of one of formulas (III-1)-(III-6): 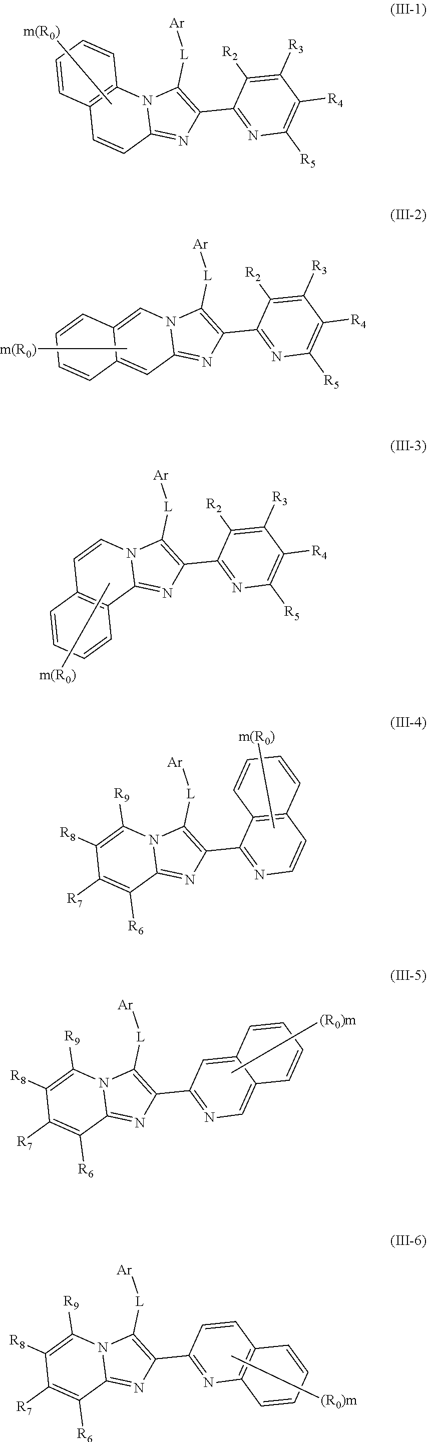
wherein, each of R₀ and R₂-R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups are capable of being fused to form a ring; each L is a substituted/unsubstituted aromatic or heteroaromatic group containing 6 to 30 ring atoms; each Ar is selected from a substituted/unsubstituted aromatic or heteroaromatic group containing 5 to 40 ring atoms, an aryloxy or heteroaryloxy group containing 5 to 40 ring atoms, or any combination thereof, wherein Ars form a monocyclic or polycyclic aliphatic or aromatic ring systems with rings bonded thereto; m is an integer from 0 to 6.

4. The organic electronic device according to claim 2, wherein the organic compound comprises a structure of one of formulas (III-1)-(III-6): 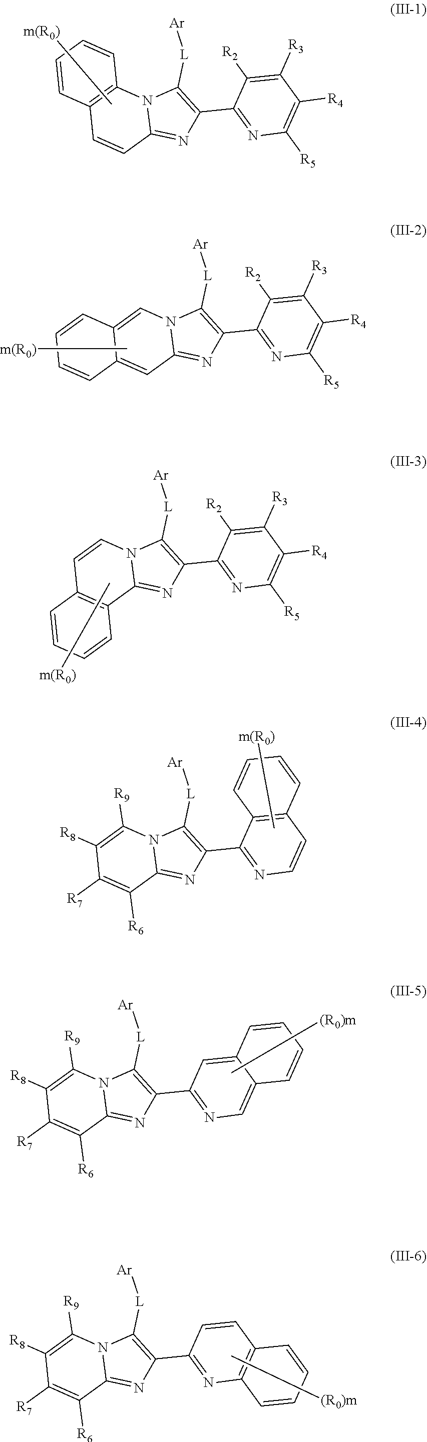
wherein, each of R₀ and R₂-R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups are capable of being fused to form a ring; each L is a substituted/unsubstituted aromatic or heteroaromatic group containing 6 to 30 ring atoms; each Ar is selected from a substituted/unsubstituted aromatic or heteroaromatic group containing 5 to 40 ring atoms, an aryloxy or heteroaryloxy group containing 5 to 40 ring atoms, or any combination thereof, wherein Ars form a monocyclic or polycyclic aliphatic or aromatic ring systems with rings bonded thereto; m is an integer from 0 to 6.

5. The organic electronic device according to claim 1, wherein each L is independently selected from the following groups or any combination thereof: 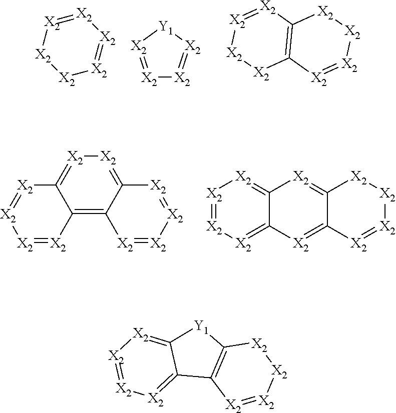
wherein:
X₂ at each occurrence is independently selected from CR₁₀ or N;
Y₁ at each occurrence is independently selected from NR₁₁, CR₁₂R₁₃, 0, S, SiR₁₄R₁₅, S═O, or SO₂;
each of R₁₀ to R₁₅ at each occurrence is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, and any combination thereof.

6. The organic electronic device according to claim 2, wherein each L is independently selected from the following groups or any combination thereof: 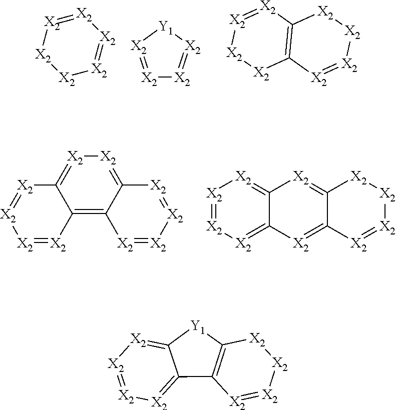
wherein:
X₂ at each occurrence is independently selected from CR₁₀ or N;
Y₁ at each occurrence is independently selected from NR₁₁, CR₁₂R₁₃, O, S, SiR₁₄R₁₅, S═O, or SO₂;
each of R₁₀ to R₁₅ at each occurrence is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, and any combination thereof.

7. The organic electronic device according to claim 3, wherein each L is independently selected from the following groups or any combination thereof: 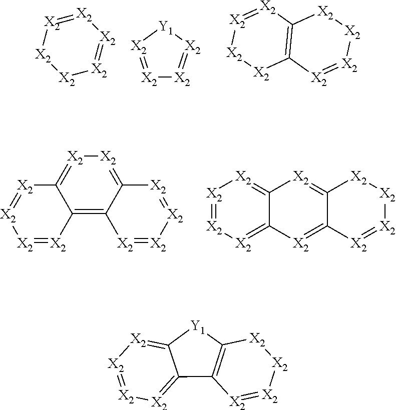
wherein:
X₂ at each occurrence is independently selected from CR₁₀ or N;
Y₁ at each occurrence is independently selected from NR₁₁, CR₁₂R₁₃, O, S, SiR₁₄R₁₅, S═O, or SO₂;
each of R₁₀ to R₁₅ at each occurrence is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, and any combination thereof.

8. The organic electronic device according to claim 4, wherein each L is independently selected from the following groups or any combination thereof: 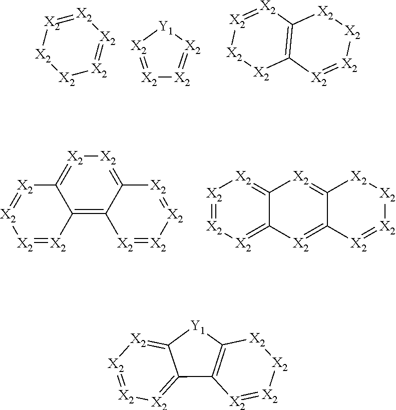
wherein:
X₂ at each occurrence is independently selected from CR₁₀ or N;
Y₁ at each occurrence is independently selected from NR₁₁, CR₁₂R₁₃, O, S, SiR₁₄R₁₅, S═O, or SO₂;
each of R₁₀ to R₁₅ at each occurrence is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, and any combination thereof.

9. The organic electronic device according to claim 1, wherein each L is independently selected from the following groups or any combination thereof:

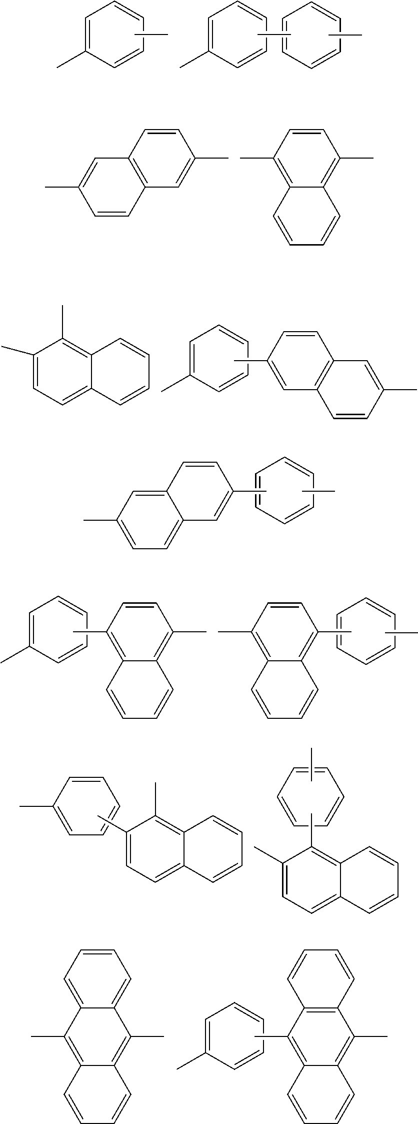

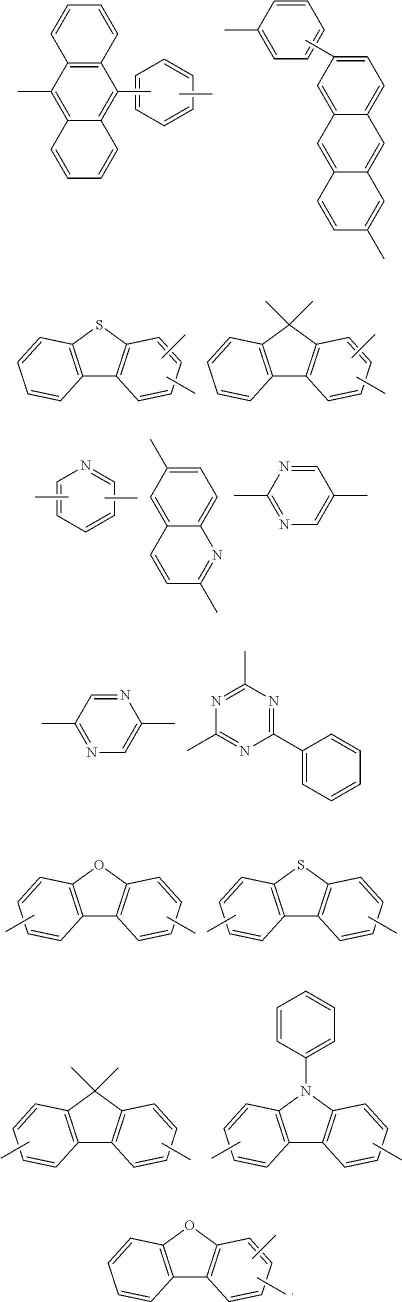

10. The organic electronic device according to claim 2, wherein each L is independently selected from the following groups or any combination thereof:

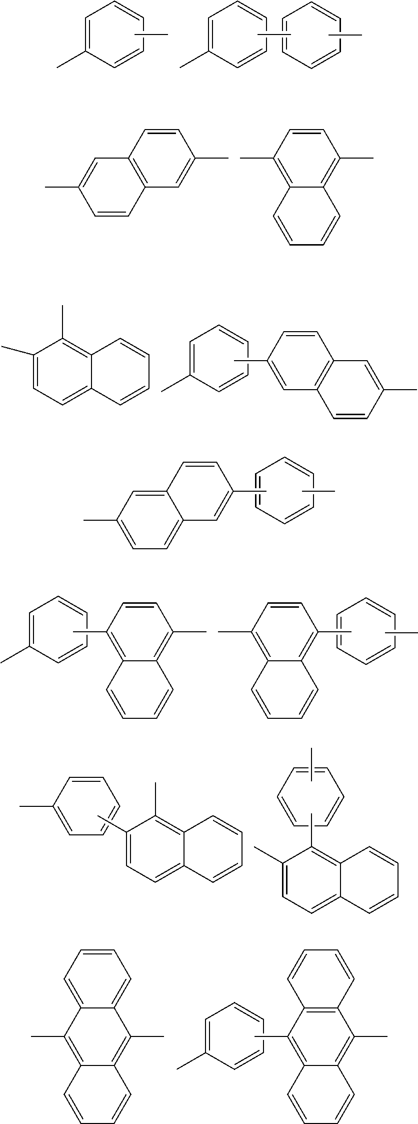

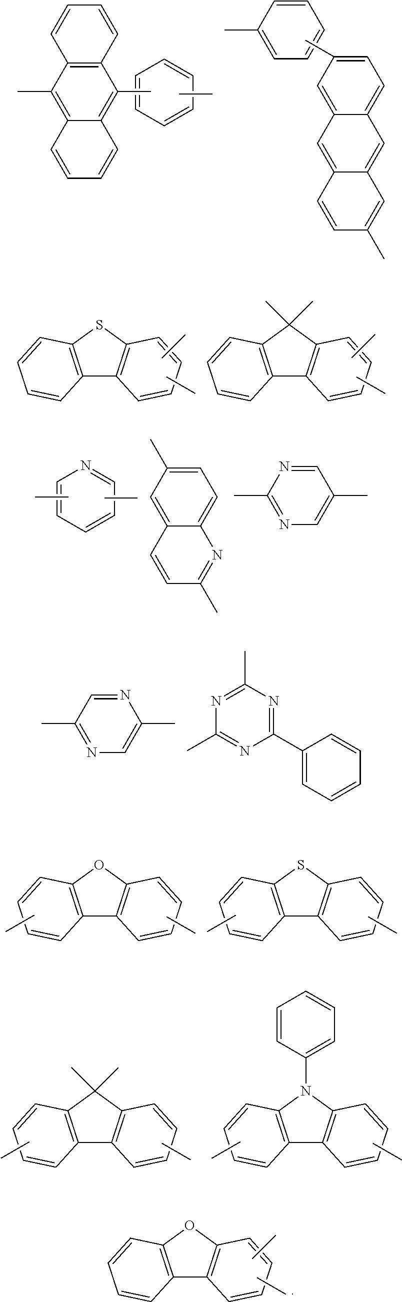

11. The organic electronic device according to claim 3, wherein each L is independently selected from the following groups or any combination thereof:

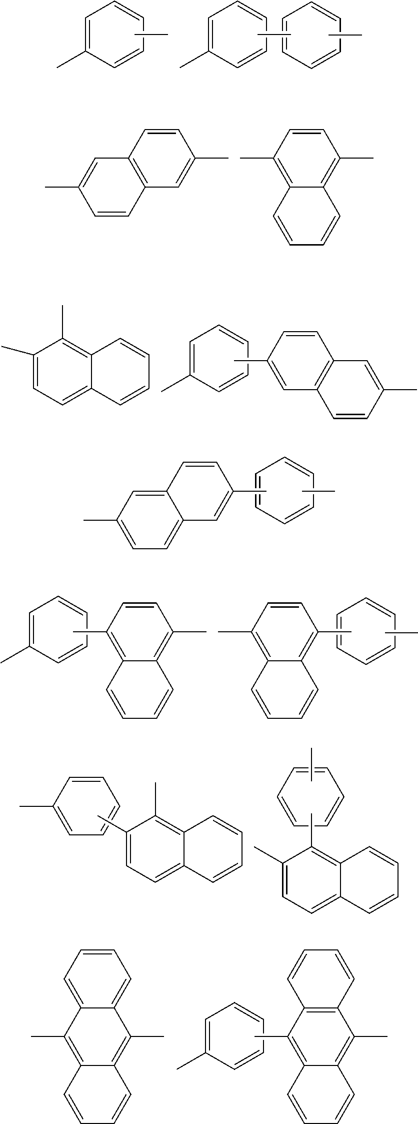

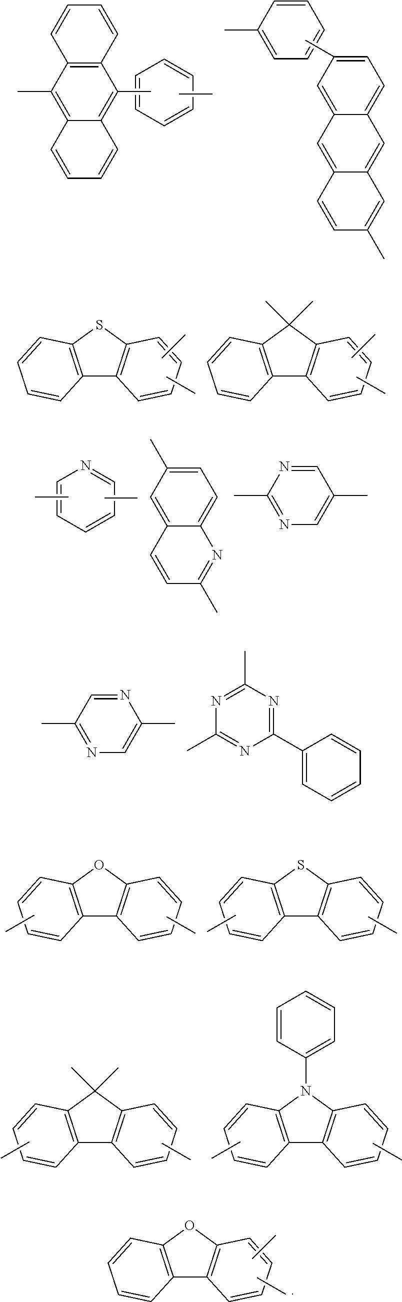

12. The organic electronic device according to claim 4, wherein each L is independently selected from the following groups or any combination thereof:

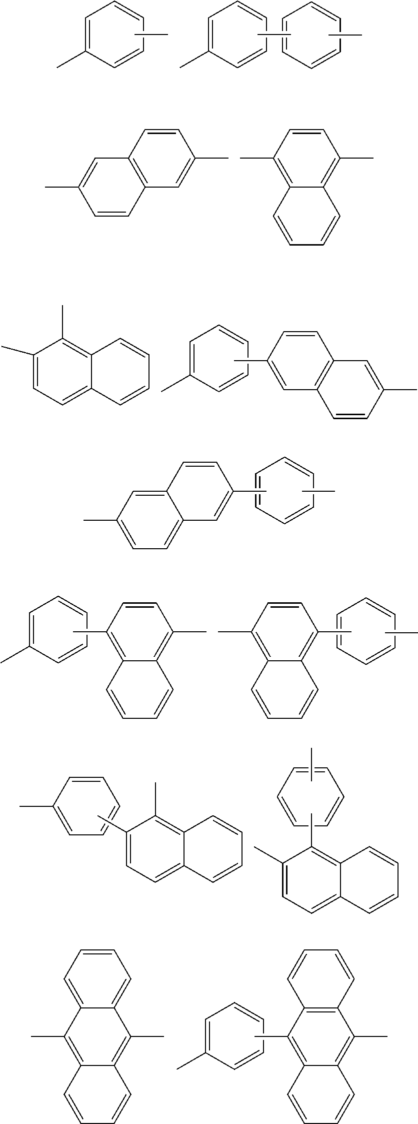

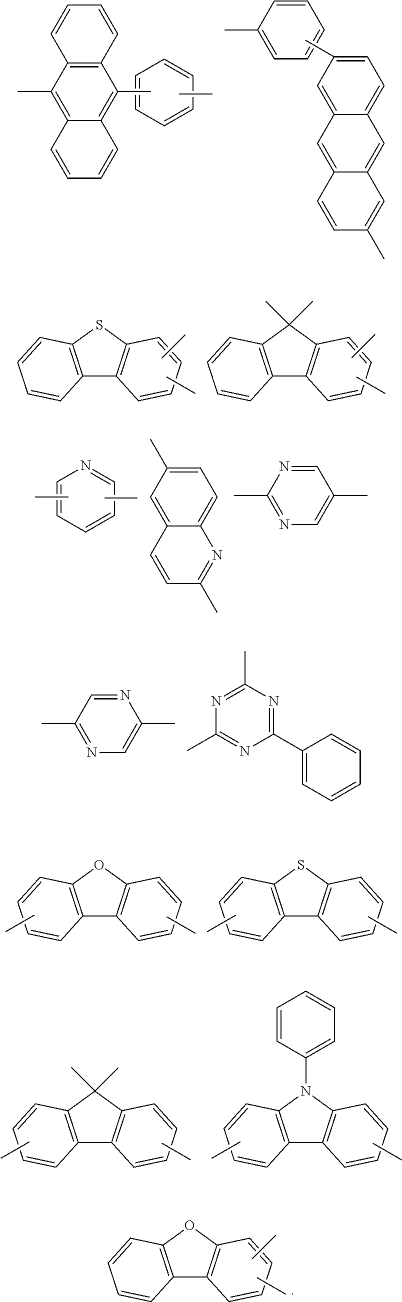

13. The organic electronic device according to claim 5, wherein each L is independently selected from the following groups or any combination thereof:

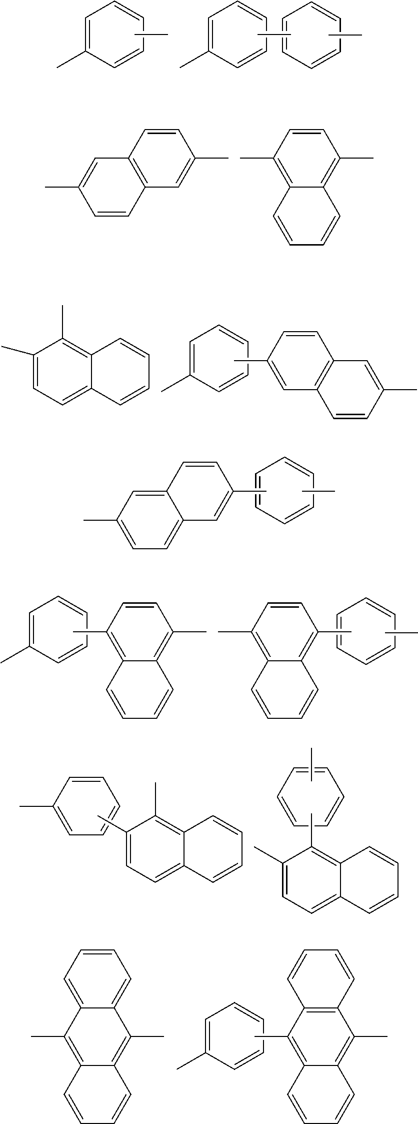

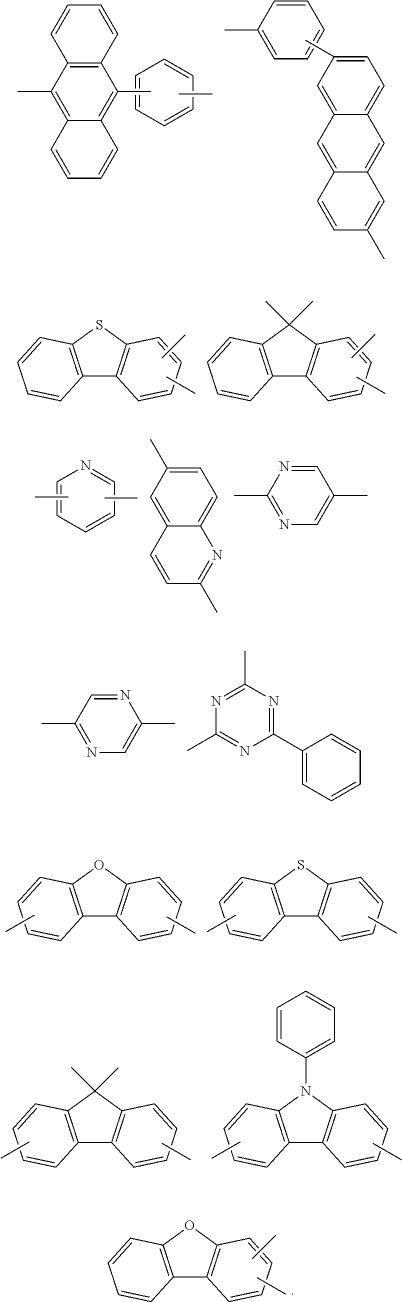

14. The organic electronic device according to claim 6, wherein each L is independently selected from the following groups or any combination thereof:

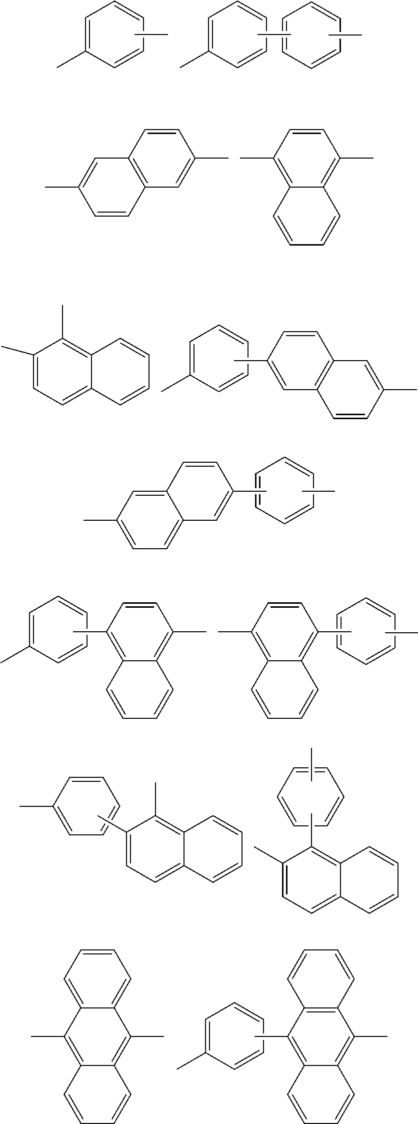

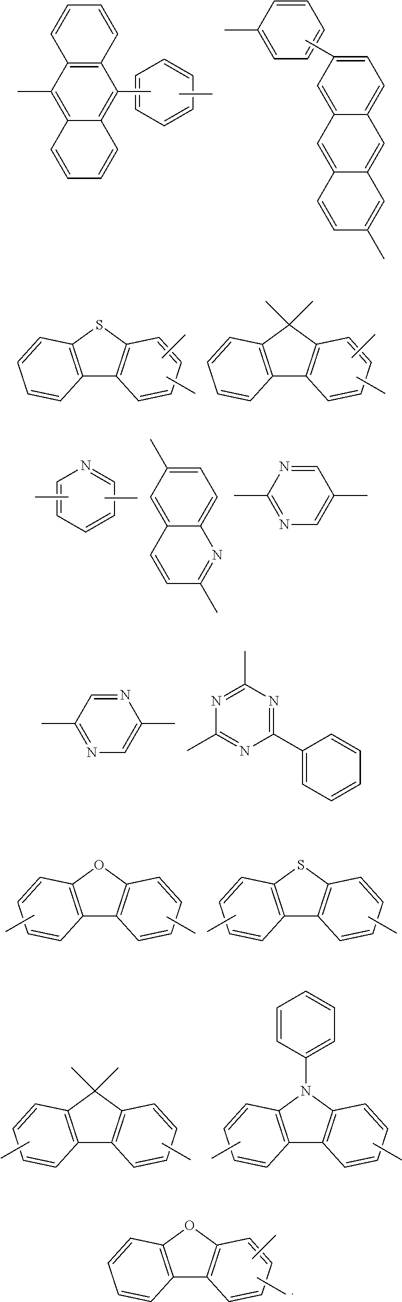

15. The organic electronic device according to claim 7, wherein each L is independently selected from the following groups or any combination thereof:

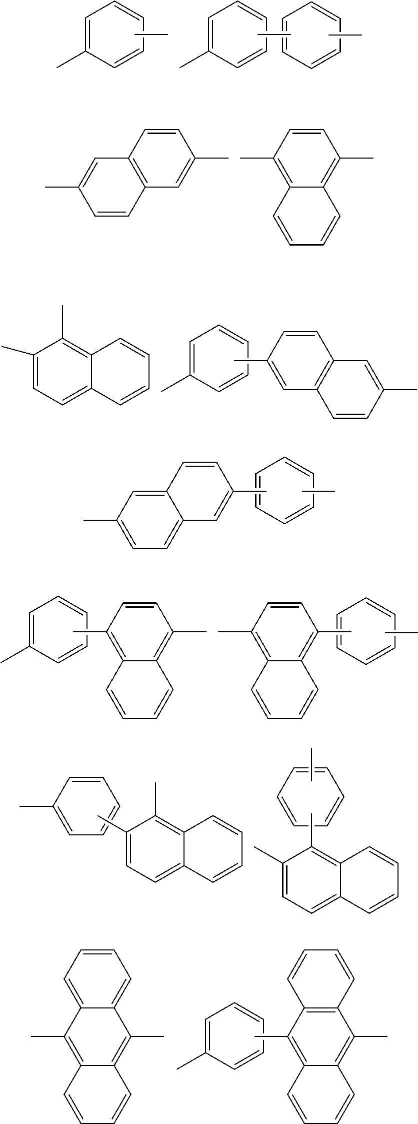

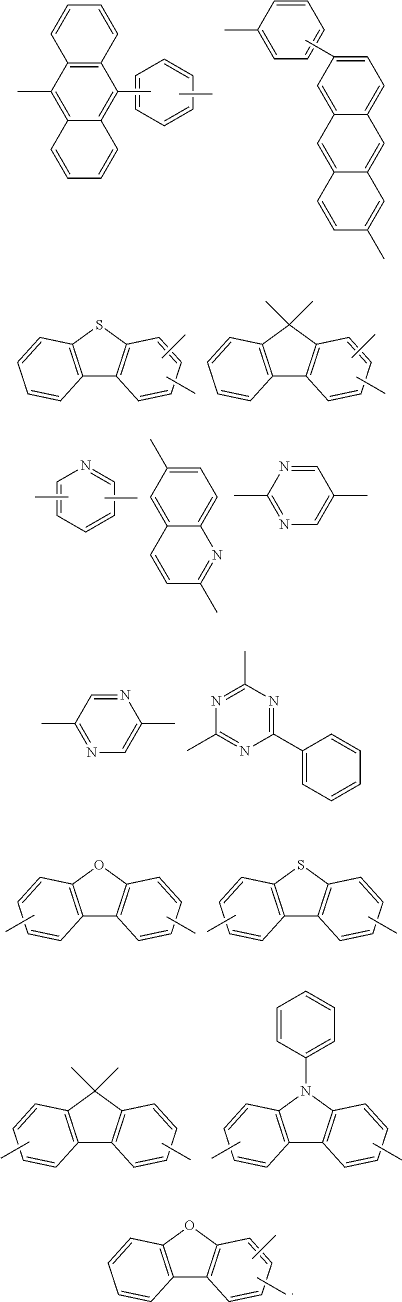

16. The organic electronic device according to claim 8, wherein each L is independently selected from the following groups or any combination thereof:

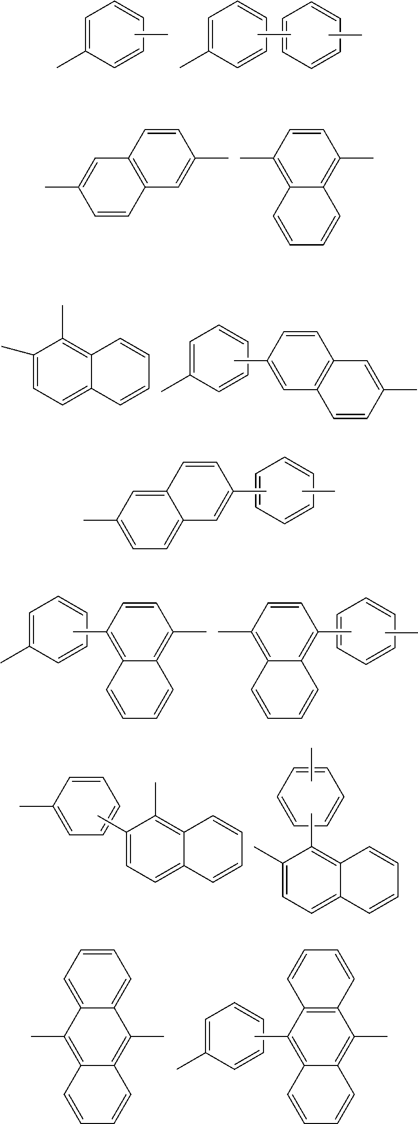

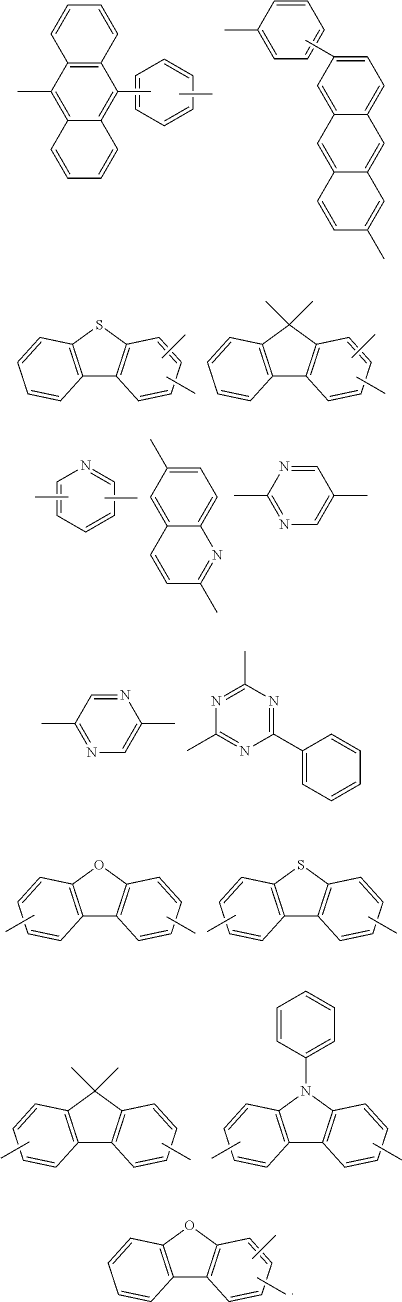

17. The organic electronic device according to claim 1, wherein each Ar comprises an electron-withdrawing group.

18. The organic electronic device according to claim 17, wherein the electron-withdrawing group is selected from F, a cyano group, one of the following groups, or any combination thereof: 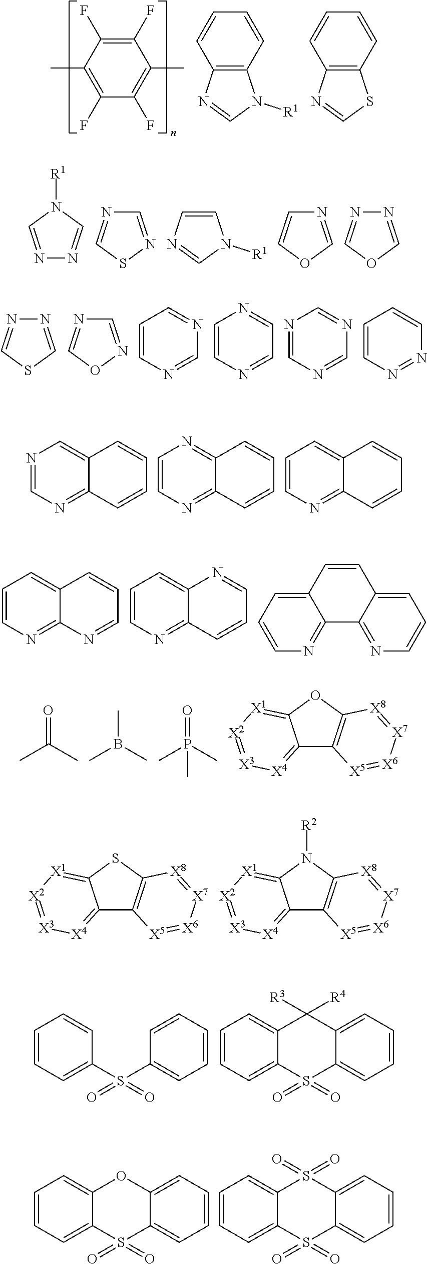 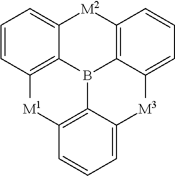
wherein n is 1, or 2, or 3; each of X¹ to X⁸ is CR⁵ or N, and at least one of them is N; meanwhile any two adjacent positions form a monocyclic or polycyclic aliphatic or aromatic ring system; M¹, M², M³ independently represent N(R⁶), C(R⁷)₂, Si(R⁸)₂, O, C═N(R⁹), C═C(R¹⁰)₂, P(R¹¹), P(═O)R¹², S, S═O, SO₂, or null; each of R¹ to R¹² at each occurrence is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, and any combination thereof.

19. A compound, comprising a structure of formula (1): 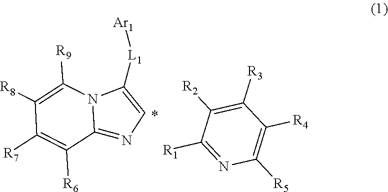
wherein, any one of R₁-R₅ is directly linked to * via a single bond;
each of R₁ to R₉ is a substituent, and at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof, and the adjacent two groups are capable of being fused to form a ring;
each L₁ is a substituted/unsubstituted aromatic group containing 6 to 30 ring atoms;
each Ar₁ is selected from a substituted/unsubstituted aromatic or heteroaromatic group containing 5 to 40 ring atoms, an aryloxy or heteroaryloxy group containing 5 to 40 ring atoms, or any combination thereof, wherein Ar₁s form a monocyclic or polycyclic aliphatic or aromatic ring systems with rings bonded thereto, and the ring atom of Ar₁ is directly linked to the ring atom of L₁ via a single bond.

20. An organic electronic device, comprising at least one of the compound according to claim 19, wherein the organic electronic device is preferably selected from an organic light emitting diode, an organic photovoltaic cell, an organic light emitting electrochemical cell, an organic field effect transistor, an organic light emitting field effect transistor, an organic laser, an organic spintronic electronic device, an organic sensor, or an organic plasmon emitting diode.
